(19) United States
(12) Patent Application Publication
(10) Pub. No.: US 2026/0234607 A1
(43) Pub. Date: Aug. 13, 2026

(54) METHOD OF MULTI-LOCUS CRISPRI TARGETING WITH A SINGLE TRUNCATED GUIDE

(71) Applicant: The Broad Institute, Inc., Cambridge, MA (US)

(72) Inventors: Fadi NAJM, Cambridge, MA (US); Molly MOORE, Cambridge, MA (US)

(73) Assignee: The Broad Institute, Inc., Cambridge, MA (US)

(21) Appl. No.: 19/651,452

(22) Filed: Apr. 17, 2026

Related U.S. Application Data
(63) Continuation of application No. PCT/US2024/051376, filed on Oct. 15, 2024.
(60) Provisional application No. 63/591,610, filed on Oct. 19, 2023.

(51) Int. Cl. C12N 15/10; C12N 9/22; C12N 15/11; C12N 15/86

(52) U.S. Cl. CPC C12N 15/1086; C12N 9/226; C12N 15/1082; C12N 15/111; C12N 15/86; C07K 2319/71; C07K 2319/81; C12N 2310/20; C12N 2750/14143

(57) ABSTRACT

Provided herein are methods and compositions for screening of multiple nucleic acid sites, such as functional non-coding transcriptional regulatory elements in a cell. In particular, provided are methods and compositions, comprising: delivering to a cell a composition comprising: a single guide RNA (sgRNA) or a polynucleotide encoding the sgRNA comprising a sequence capable of hybridizing with a target sequence present in a non-coding transcriptional regulatory element, and an effector protein or one or more nucleotide sequences encoding the effector protein optionally comprising an effector domain, wherein the sgRNA hybridizes to the target sequence and forms a complex with the effector protein; and measuring gene expression upon binding of the complex to the target sequence.

### CROSS-REFERENCE TO RELATED APPLICATIONS

This application is a continuation of International Application No. PCT/US2024/051376, filed on Oct. 15, 2024, and claims the benefit under 35 U.S.C. § 119(e) of U.S. Provisional Patent Application No. 63/591,610, filed Oct. 19, 2023, the entire contents of which is incorporated herein by reference in its entirety.

### TECHNICAL FIELD

The present invention generally relates to systems, methods and compositions used for the control of gene expression involving sequence targeting, such as perturbation of gene transcripts or nucleic acid editing, that may use vector systems related to Clustered Regularly Interspaced Short Palindromic Repeats (CRISPR) and components thereof.

### BACKGROUND

A critical goal in functional genomics is evaluating which non-coding elements contribute to gene expression, cellular function, and disease. Functional characterization remains a challenge due to the abundance and complexity of candidate elements. To date, over 1 million human cis-regulatory elements (CREs) have been cataloged across various cell and tissue types. CREs include the promoters, enhancers, insulators, and silencers that direct gene expression, sometimes in dynamic interplay or synergy. CRE function is further influenced by cell specific states and transcription factor (TF) binding sites. TFs recruit proteins and complexes to orchestrate gene expression. TFs bind with various strengths, often dictated by cell state and genomic contexts such as motif combinations and orientations. However, the determinants for TF binding to one motif over another and the effect of that binding are not well understood. Connecting CREs and TF binding with functional outputs can explain variant outcomes or nominate regions for clinical interventions. Together, TFs and CREs direct the intricate regulatory networks that govern cell function and disease.

Precise genome technologies are needed to enable systematic high throughput targeting and analysis of CREs to allow for selective perturbation of individual genetic elements, as well as to advance synthetic biology, biotechnological, and medical applications. Although genome-editing techniques such as designer zinc fingers, transcription activator-like effectors (TALEs), or homing meganucleases are available for producing targeted genome perturbations, there remains a need for new genome engineering technologies that employ novel strategies and molecular mechanisms and are affordable, easy to set up, scalable, and amenable to targeting multiple positions within the eukaryotic genome. This would provide a major resource for new applications in genome engineering and biotechnology.

The present disclosure provides a CRISPRi-based approach for multi-locus screening of putative transcription factor binding sites with a single truncated guide. This approach can be valuable for elucidating functional transcription factor binding motifs or other repeated genomic sequences and is easily implementable with existing tools.

### SUMMARY

The present disclosure provides CRISPRi-based methods for targeting non-coding regulatory elements with truncated guides.

In one aspect, the present disclosure provides a high-throughput method for determining functional, non-coding transcriptional regulatory elements, comprising: a) delivering to a cell a composition comprising: i) a single guide RNA (sgRNA) or a polynucleotide encoding the sgRNA, wherein the sgRNA comprises a sequence capable of hybridizing with a target sequence; and ii) an effector protein or one or more nucleotide sequences encoding the effector protein, wherein the effector protein optionally comprises an effector domain; wherein the sgRNA hybridizes to the target sequence and forms a complex with the effector protein; and b) measuring gene expression upon binding of the complex to the target sequence.

In another aspect, the present disclosure provides an engineered, non-naturally occurring composition, comprising: a single guide RNA (sgRNA) which comprises a sequence capable of hybridizing with a target sequence, or a polynucleotide encoding the sgRNA, and an effector protein, or one or more nucleotide sequences encoding the effector protein; wherein the sgRNA hybridizes to said target sequence, and the sgRNA forms a complex with the effector protein; wherein the effector protein optionally comprises an effector domain, and wherein the sgRNA is capable of hybridizing multiple transcription factor binding sites.

In another aspect, the present disclosure provides a single guide RNA (sgRNA) comprising a nucleotide sequence as set forth in any one of SEQ ID NO: 1-46 and 50-192.

In another aspect, the present disclosure provides an adeno-associated virus (AAV) particle comprising a composition, or sgRNA described herein.

In another aspect, the present disclosure provides vector system comprising one or more vectors, wherein the one or more vectors comprises: a) a first expression regulatory element operably linked to a nucleotide sequence encoding an effector protein, or one or more nucleotide sequences encoding the effector protein; and b) a second expression regulatory element operably linked to one or more nucleotide sequences encoding a single guide RNA (sgRNA) comprising a sequence capable of hybridizing to a target sequence, wherein components (a) and (b) are located on same or different vectors, and wherein the sgRNA is capable of hybridizing to one or more non-coding transcriptional regulatory elements.

In some embodiments, the target sequence is a repeated nucleic acid sequence. In some embodiments, the repeated nucleic acid sequence is present in a promoter, an enhancer, a transposable element, a 5′UTR, or an intron.

In some embodiments, the target sequence is a transcription factor binding sequence. In some embodiments, the transcription factor binding sequence binds a transcription factor from the Ets tryptophan cluster factors family of transcription factors, the C2H2 zinc finger factors family of transcription factors, or the nuclear receptors with C4 zinc fingers family of transcription factors.

In some embodiments, the transcription factor is selected from the group consisting of AhR:Arnt, AIRE, alpha-CP1, Alx-4, AML AP-2, AP-2alphaA, AR, AREB6, ATF6, BCL6, Brachyury, CACCC-binding factor, CDP, c-Ets-1 p54, c-Myc, COUP direct repeat 1, CP2/LBP-1c/LSF, c-Rel, CCCTC-binding factor (CTCF), CTF1, deltaEF1, E12, E2F-1, E47, EBF, Egr-1, Egr-3, Elf-1, Elk-1, ER, Ets, ETS2, FXR inverted repeat 1, FXR/RXR-alpha, GABP, GAF, GCM, GLI, GLI1, Hand1:E47, HEB, Helios A, HEN1, HNF4 direct repeat 1, HNF4, COUP, Ikaros, KAISO, LM4_M2, LRF, LRH1, LUN-1, LXR, LXR direct repeat 4, LXR, PXR, CAR, COUP, RAR, Lyf-1, MAF, MIF-1, MIZF, MyoD, myogenin/NF-1, NERF1a, NF-1, NF-AT, NF-kappaB, NF-kappaB (p50), NF-kappaB (p65), NF-muE1, NF-Y, NGFI-C, NRSF, NUR77, Olf-1, P50: RELA-P65, p53, Pax, Pax-1, Pax-6, PEBP, Pitx2, PPAR, PPAR direct repeat 1, PPARalpha:RXRalpha, PPARgamma:RXRalpha, PPARgamma, PU.1, PXR, CAR, LXR, FXR, RBP-Jkappa, RelB:p52 (NF-kappaB), REST, RFX1, Roaz, RORalpha, RORalpha1, RORalpha2, RP58, SAP-1a, SF1, Sp3, SREBP, SREBP-1, SRF, Staf, STAT1, STAT3, STAT5A (homodimer), STAT5B (homodimer), STATx, SZF1-1, TAL1, Tax/CREB, TBX22, TBX5, Tel-2, VDR, YY1, ZBRK1, Zic2, and ZID.

In some embodiments, the sgRNA is 9nt-21nt in length. In some embodiments, the sgRNA is 9nt-14nt in length. In some embodiments, the sgRNA is 9nt in length. In some embodiments, the sgRNA comprises 0-5 mismatched base pairs with the target sequence.

In some embodiments, the effector protein comprises a zinc finger nuclease, a TALEN, a Cas protein, Cpf1 (Cas12), C2c2 (Cas13), Csm/Cmr, C2c6, Csy4, Csm6, C2c2c, dC2c, or Fanzor. In some embodiments, the method comprises a Clustered Regularly Interspaced Short Palindromic Repeats (CRISPR)-associated (CRISPR-Cas) system.

In some embodiments, the effector domain is selected from the group consisting of: transposase domain, integrase domain, recombinase domain, resolvase domain, invertase domain, protease domain, DNA methyltransferase domain, DNA hydroxylmethylase domain, DNA demethylase domain, histone acetylase domain, histone deacetylases domain, nuclease domain, repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase, and histone tail protease.

In some embodiments, the effector protein comprises a Cas protein fused to a repression domain. In some embodiments, the repression domain is selected from the group consisting of KRAB, DNMT1, HDAC, DNMT3A, DNMT3A/3L, MeCP2, SID, NuRD, HP1, LSD1, SETDB1, EZH2, or a combination thereof.

In some embodiments, the effector protein comprises a Cas protein fused to an activator domain. In some embodiments, the activator domain is selected from the group consisting of VP64, p65, VPR (VP64-p65-Rta), p65-HSF1, VPR-RecA, or a combination thereof.

In some embodiments, the effector protein is a catalytically inactive Cas 9 (dCas9).

In some embodiments, the target sequence is a CTCF, PU.1, SP1, YY1, or NR2 binding sequence.

In some embodiments, the sgRNA comprises a nucleotide sequence as set forth in any one of SEQ ID Nos: 1-46 and 50-192.

In another aspect, the present disclosure provides an isolated cell comprising a composition, sgRNA, AAV particle, or vector system described herein.

In another aspect, the present disclosure provides an in vitro or ex vivo host cell or cell line or progeny thereof, comprising a composition, sgRNA, AAV particle, or vector system disclosed herein.

In another aspect, the present disclosure provides methods for screening for functional non-coding transcriptional regulatory elements in a cell, the method comprises delivering to the cell a composition, sgRNA, AAV, or vector system disclosed herein, wherein the effector protein forms a complex with the sgRNA and upon binding of the complex to a target sequence, a modification in gene expression occurs. In some embodiments, gene expression is increased or decreased upon binding of the complex to a target sequence.

This summary is illustrative only and is not intended to be in any way limiting. Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention. These and other embodiments are disclosed or are obvious from and encompassed by, the following Detailed Description.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows truncated guides enable on-target CRISPRi-mediated repression. A Schematic of truncated guide experiments. B Convention for guide cloning design with target matching sequence highlighted in gray. Guide sequences tested are based on 5′ truncations in sgCD81i-1, that targets the CD81 TSS. C CRISPRi in Jurkat cells with truncated CD81 promoter targeting guide and analyzed for CD81 cell surface expression. D CRISPRi treatment of two additional CD81 TSS targeting guides and respective 5′ truncations. CD81 expression was read by flow cytometry. E Cas9 cleavage in Jurkat cells treated with two guides targeting CD81 and truncated versions. For C-E, cells were stained with CD81-FITC antibody analyzed by flow cytometry 7 days post lentiviral transduction for CD81 targeting guide, safe harbor control (Safe), or untransduced (UT). Flow gating strategy found in FIG. 2E. Data are mean±/−SD from biological triplicate.

FIG. 2 shows specificity of truncated guide repression. A Jurkat and B A375 cells CRISPRi treated with the CD81i-1 based guides in FIG. 1B and analyzed for CD81 expression by flow cytometry. C CRISPRi in A375 cells with truncated CD81 promoter targeting guide and analyzed for CD81 cell surface expression. For A-C, cells were stained with CD81-FITC antibody analyzed by flow cytometry 7 days post lentiviral transduction for CD81 targeting guide, safe harbor control (Safe), or untransduced (UT). Data are mean±/−SD from biological triplicate. D Gene expression in A375 cells in 20nt and g[9nt] sgCD81i-1 CRISPRi populations. p-value cutoff at >2 and log fold-change >2 and <−2. E Gating strategy for live cell selection (left panel) and CD81+ selection by setting the gate <0.1% positive in untreated isotype control stained cells (right panel).

FIG. 3 shows that enhancer targeting with truncated guides induces EPB41 repression. A Schematic of EPB41 genomic regulatory region and full-length/truncated guides targeting each of 4 TF motifs. B TF motifs (JASPAR) targeted by the guides with protospacer adjacent motif (PAM) orientation denoted. C Quantitative real time PCR analysis of EPB41 expression in K562 cells with respective guide CRISPRi treatment conditions. Safe (Safe Harbor-targeting) guide=g[20nt], promoter guide=21nt, enh1=g[20nt], enh2=21nt, enh3=g[20nt]. Data are mean+/−SEM. D Violin plot summary of EPB41 expression data in panel C organized by guide length or targeting region. TF-targeting guides (n=16; 4 guides per spacer length), sgEnh (n=12; 3 guides), and Promoter (n=4; 1 guide, all points shown) are the product of 2 biological replicates per guide run in duplicate. Safe is the sum of 3 biological replicates run 2-3 times each (n=8). Data are normalized to the mean CT value of Safe Harbor actin-b control probe. Results are analyzed by one-way ANOVA. p****<0.0001 for all guide lengths compared with Safe Harbor guide.

FIG. 4 shows simultaneous screening of CCCTC-binding factor (“CTCF)” binding sites with a truncated guide. A Guide selection strategy based on top Homer generated motif from CTCF ChIP in Jurkat cells. B CTCF targeting schematic of a single 10nt guide directing CRISPRi to many motifs simultaneously. C Target site distribution and CTCF occupancy based on Jurkat ChIP for each of the 10nt guide sequences. D CTCF ChIPseq data depicting the fraction of perfect match sites with CTCF bound in A375 cells for each guide in the CTCF library. E Fraction of CTCF ChIPseq sites targeted by the 24 guide library in Jurkat (left) and A375 (right) cells.

FIG. 5 shows CTCF library analysis. A CTCF promoter targeting guide fitness in the 5 cell lines tested (indicated). B, C Guide enrichment and depletion in pooled screen for the indicated cell lines. CTCF promoter targeting full length guide knockdown (KD) used as a control. Pooled screens were run in triplicate, while K562 was in duplicate. Data are mean±/−SD.

FIG. 6 shows that appending an 11th nucleotide to the CTCF library guides. Left, pearson correlations for the n[10nt] screening data compared to the respective 10nt guide data for the indicated cell line. Right, fitness effects of each n[10nt] guide for each of the cell lines tested. Z-score calculated from log fold change relative to safe harbor control. Experiments were run in triplicate except for K562, in duplicate.

FIG. 7 shows CRISPRi with a single truncated guide disrupts CTCF binding at multiple loci. A CTCF ChIP of the CTCF bound sites (#) targeted by sg4 with a perfect match. B Scatter plot CTCF binding at JASPAR motifs (880k) with 357 perfect match sites (black) and partial match sites (grey). C CTCF ChIPseq density plot and heatmaps of sg4 and aag[sg4] targeted loci with a 6 kb window for Jurkat cells treated with sg4, aag[sg4], or Safe Harbor (SH) guides. Top heatmap cluster represents sites with an aag[sg4] sequence match, which sg4 also matches, and the bottom heatmap cluster are sites only sg4 has a sequence match.

FIG. 8 shows a correlation between CTCF loss and an increase in H3K9me3 signal at perfect match sites. A H3K9me3 ChIP of all sg4 perfect match sites (357). B H3K9me3 density plot for Safe Harbor (S.H.) and sg4 treated Jurkat cells. C, D Track views of 2 targeted regions depicting CTCF and H3K9me3 signal from 2 replicate experiments (rep). Bars on the bottom depict CTCF perfect match (black) and partial match sites (grey).

FIG. 9 shows CTCF targeting with aag[sg4]. A scatter plots of CTCF binding at JASPAR motifs (880k) with perfect match sites (black), aag[sg4] (13nt) sites, and partial match sites. B boxplot of CTCF binding at 26 genomic loci targeted by both sg4 and aag[sg4]. t-test ***p 2.16×10⁻⁶ for sg4 and ***p 1.48×10⁻⁸ for aag[sg4]. Comparison between sg4 and aag[sg4] was not significant (NS).

FIG. 10 shows, targeting data of sg8. A Boxplot of CTCF signal at bound sequence match sites of sg8 and safe harbor treated Jurkat cells. B Boxplot of H3k9me3 signal at sequence match sites. C Scatter plot of JASPAR annotated CTCF sites with sg8 perfect match sites (black) and all other sites (grey) in rep1 and rep 2 Jurkat cells.

FIG. 11 shows gene expression in CTCF truncated guides. Jurkat cells treated with sg4, sg8, or safe harbor targeting CRISPRi lentivirus for 7 days and processed by RNAseq. A, B Volcano plots of sg4 (A) and sg8 (B) normalized to safe harbor. Dotted lines represent thresholds set at log fold change [abs 2] and −log (P-value)>3. C, D Heatmaps of the top differential genes in sg4, none significant (C) and the top 67 significant differential genes in sg8 (D) alongside safe harbor control gene expression.

FIG. 12 shows analysis of significantly disrupted CTCF sites. A Volcano plot of CTCF bound sg4 sites (400k) with significant 10nt match CTCF sites, significant partial match sites (79), and not significant sites. Significance was determined at cutoffs (abs [LFC]>0.5 and −log 10 pval>5). B Histogram depicting significant CTCF disrupted sites as a fraction of CTCF bound sites with 0 mismatches up to 5 mismatches. Raw values depicted on each bar. C Volcano plot of significantly targeted sites determined by dream analysis with sg8 perfect match sites (black) and all other sites (gray). D, E Logogram of significant perfect match sites (top) and significant partial match sites (bottom) from sg4 experiments (D) and sg8 experiments (E).

### DETAILED DESCRIPTION

Before the present methods of the invention are described, it is to be understood that this invention is not limited to particular methods, components, products or combinations described, as such methods, components, products and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”. It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention. It may be advantageous in the practice of the invention to be in compliance with Art. 53(c) EPC and Rule 28(b) and (c) EPC. Nothing herein is intended as a promise.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Standard reference works setting forth the general principles of recombinant DNA technology include Molecular Cloning: A Laboratory Manual, 2nd ed., vol. 1-3, ed. Sambrook et al., Cold Spring Harbor Laboratory Press, Cold Spring Harbor, N.Y., 1989; Current Protocols in Molecular Biology, ed. Ausubel et al., Greene Publishing and Wiley-Interscience, New York, 1992 (with periodic updates) (“Ausubel et al. 1992”); the series Methods in Enzymology (Academic Press, Inc.); Innis et al., PCR Protocols: A Guide to Methods and Applications, Academic Press: San Diego, 1990; PCR 2: A Practical Approach (M.J. MacPherson, B.D. Hames and G. R. Taylor eds. (1995); Harlow and Lane, eds. (1988) Antibodies, a Laboratory Manual; and Animal Cell Culture (R.I. Freshney, ed. (1987). General principles of microbiology are set forth, for example, in Davis, B. D. et al., Microbiology, 3rd edition, Harper & Row, publishers, Philadelphia, Pa. (1980).

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In this description of the invention, reference is made to the accompanying drawings that form a part hereof, and in which are shown by way of illustration only of specific embodiments in which the invention may be practiced. It is to be understood that other embodiments may be utilised and structural or logical changes may be made without departing from the scope of the present invention. The description, therefore, is not to be taken in a limiting sense, and the scope of the present invention is defined by the appended claims.

It is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product.

Preferred statements (features) and embodiments of this invention are set herein below. Each statements and embodiments of the invention so defined may be combined with any other statement and/or embodiments unless clearly indicated to the contrary. In particular, any feature indicated as being preferred or advantageous may be combined with any other feature or features or statements indicated as being preferred or advantageous.

### I. Introduction

The present disclosure relates to the effective CRISPR interference (CRISPRi) targeting at multiple genomic loci with truncated guide RNAs (tr-sgRNA). This disclosure also provides methods of characterization of non-coding regulatory elements, including promoters, enhancers, insulators, and silencers, that direct gene expression. A key advantage of the present invention, is the ability to control the specificity of the guides and make use of the promiscuity of the CRISPR system by changing the length of the guides.

Over 1 million human cis-regulatory elements (CREs) have been cataloged across various cell and tissue types (ENCODE Project Consortium et al. (2020) Nature, 583(7818):699-710; Gerstein et al. (2012) Nature, 489(7414):91-100); Roadmap Epigenomics Consortium et al. (2015) Nature, 518(7539):317-330; The ENCODE Project Consortium (2012) Nature, 489(7414):57-74). CREs include the promoters, enhancers, insulators, and silencers that direct gene expression, sometimes in dynamic interplay or synergy. CRE function is further influenced by cell specific states and transcription factor (TF) binding sites. TFs recruit proteins and complexes to orchestrate gene expression. TFs bind with various strengths, often dictated by cell state and genomic contexts such as motif combinations and orientations (Amit et al. (2009) Science, 326(5950):257-263; Avsec (2021) Nature Genetics, 53(3):354-366; Zeitlinger (2020) Current Opinions in Systems Biology, 23:22-31). However, the determinants for TF binding to one motif over another and the effect of that binding are not well understood. Connecting CREs and TF binding with functional outputs can explain variant outcomes or nominate regions for clinical interventions. Together, TFs and CREs direct the intricate regulatory networks that govern cell function and disease.

Described herein are methods that generate and employ tr-sgRNA to target dCas9 to hundreds of TF binding sites, thus expediting the discovery of functional regulatory elements. A single truncated guide can target dCas9 to hundreds of TF binding sites, thus expediting the discovery of functional regulatory elements. While specific TF binding sites were employed in the non-limiting examples provided herein, the provided methods can be used for any TF binding site or CRE.

In the invention described herein, effective CRISPRi targeting is performed at multiple genomic loci with truncated guides as short as 9nt. This is a unique property of dCas9 moieties, as catalytically active Cas9 is incapable of on-target cleavage with guides shorter than 17nt. In the non-limiting examples described herein, a library of 24 10nt guides enabled screening of over 13,000 CCCTC-binding factor (“CTCF”) sites, representing 10.8% and 14% of bound CTCF sites in Jurkat and A375 cells respectively, demonstrating scalable utility. Chromatin binding analysis revealed simultaneous disruption of multiple CTCF binding events with a single truncated guide at most sequence match sites.

The activity of a guide depends on seed sequence, which impacts best outcomes of both 20nt and 10nt guides. When designing a truncated guide library, it is important to consider exact seed sequence as some TF motifs will be better targeted than others. PAM distal mismatches of a few bases are tolerated with full-length guide-directed dCas9 (Boyle et al. (2017) PNAS, 114(21):5461-5466) and the same 5′ flexibility with 10⁻¹³nt guides was observed in the examples provided herein. There are considerations beyond the guide sequence that determine dCas9 targeting efficiency. Furthermore, it is unclear how required H3K9me3 is for disruption of CTCF binding. A recent study demonstrated better enhancer and promoter targeting with a KRAB-dCas9-MeCP2 system than with KRAB-dCas9 alone (Morris et al. (2023) Science, 380(6646):eadh7699), suggesting the importance of the repressive marks. In this work TF binding motifs directly were targeted, making it plausible that steric hindrance contributes to TF displacement from chromatin.

Selection of targeted TF motifs and guide length are critical when planning truncated guide screens. In the non-limiting examples provided herein, motifs containing an NGG PAM were selected to maximize the likelihood of dCas9 binding. In some embodiments contemplated, dCas protein variants with expanded PAM requirements are utilized to expand the available target sequences. In the examples provided herein, 10nt and 13nt guides both significantly disrupted target CTCF binding, however the 13nt guide generated a greater effect size at its targets. Explanations for this are either that longer guides form a more stable R-loop structure with dCas9 resulting in a stronger binding affinity (Josephs et al. (2015) NAR, 43(18):8924-8941; Kocak et al. (2019) Nature Biotechnology, 37(6):657-666) or longer guides benefit from a more favorable ratio of KRAB-dCas9 protein units to target sites. The maximum number of sites simultaneously targetable with a single guide is yet to be determined. Here guides were investigated with hundreds of sequence match sites (sg4 and sg8), but the exact multi-locus limit will depend on specific guide sequences and expression levels of the KRAB-dCas9 construct. Lastly, it is suggested that calculating the fraction of sequence match sites bound by the target TF (via ATAC-seq, ChIP-seq, or CUT&Tag data) is performed to assess screen efficiency. In Jurkat cells, 46.6% of sequence match CTCF sites for all guides in the library were bound by the protein. The bound fraction may be lower for other TFs and DNA-binding proteins.

The truncated guide method described herein is a first pass discovery tool for targeting repeated genomic loci or repeated nucleic acid sequences, that could include promoters, transposable elements, enhancers, TF binding sites, 5′-UTRs, and introns. This is an improved approach over TFs knockout, which may result in negative fitness outcomes or lethality, such as with CTCF. Furthermore, TFs can have alternate cellular functions, such as RNA binding (Oksuz et al. (2023) Molecular Cell, 83(14), 2449-2463), which can confound TF knockout studies but not truncated guide experiments. Binding sites are partitioned by each truncated guide and reliably assayed. Another advantage to this approach is in cell models with low lentiviral efficiencies or rare cell populations, such as primary cells. A single truncated guide provides a rich landscape of tested outcomes with fewer transduction events. Finally, single cell assays will benefit from truncated guide application due to richer gene network perturbations.

### II. Definitions

As used in the description of the invention and the appended claims, the singular forms “a,” “an” and “the” are intended to include the plural forms as well, unless the context clearly indicates otherwise.

The terms “substantially” and “about” are used herein to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. When referring to a first numerical value as “substantially” or “about” the same as a second numerical value, the terms can refer to the first numerical value being within a range of variation of less than or equal to ±10% of the second numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. The terms or “acceptable,” “effective,” or “sufficient” when used to describe the selection of any components, ranges, dose forms, etc. disclosed herein intend that said component, range, dose form, etc. is suitable for the disclosed purpose.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Also as used herein, “and/or” refers to and encompasses any and all possible combinations of one or more of the associated listed items, as well as the lack of combinations when interpreted in the alternative (“or”).

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “optional” or “optionally” means that the subsequently described event or circumstance can or cannot occur, and that the description includes instances where the event or circumstance occurs and instances where it does not.

A primer pair that specifically hybridizes under stringent conditions to a target nucleic acid may hybridize to any portion of the gene. As a result, the entire gene may be amplified or a segment of the gene may be amplified, depending on the portion of the gene to which the primers hybridize.

The terms “amplification” or “amplify” as used herein include methods for copying a target nucleic acid, thereby increasing the number of copies of a selected nucleic acid sequence. Amplification may be exponential or linear. A target nucleic acid may be DNA (such as, for example, genomic DNA and cDNA) or RNA. The sequences amplified in this manner form an “amplicon.” While the exemplary methods described hereinafter relate to amplification using the polymerase chain reaction (PCR), numerous other methods are known in the art for amplification of nucleic acids (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols, Innis et al., Eds., Academic Press, San Diego, CA 1990, pp 13-20; Wharam, et al., Nucleic Acids Res. 2001 Jun. 1; 29(11):E54-E54; Hafner, et al., Biotechniques 2001 April; 30(4):852-860.

The terms “complement,” “complementary,” or “complementarity” as used herein with reference to polynucleotides (i.e., a sequence of nucleotides such as an oligonucleotide or a target nucleic acid) refer to standard Watson/Crick pairing rules. The complement of a nucleic acid sequence such that the 5′ end of one sequence is paired with the 3′ end of the other, is in “antiparallel association.” For example, the sequence “5′-A-G-T-3” is complementary to the sequence “3′-T-C-A-5′.” Certain bases not commonly found in natural nucleic acids may be included in the nucleic acids described herein; these include, for example, inosine, 7-deazaguanine, Locked Nucleic Acids (LNA), and Peptide Nucleic Acids (PNA).

Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be a sequence of RNA complementary to the DNA sequence or its complement sequence, and can also be a cDNA. The term “substantially complementary” as used herein means that two sequences specifically hybridize (defined below). The skilled artisan will understand that substantially complementary sequences need not hybridize along their entire length. A nucleic acid that is the “full complement” or that is “fully complementary” to a reference sequence consists of a nucleotide sequence that is 100% complementary (under Watson/Crick pairing rules) to the reference sequence along the entire length of the nucleic acid that is the full complement. A full complement contains no mismatches to the reference sequence.

A “fragment” in the context of a nucleic acid refers to a sequence of nucleotide residues which are at least about 5 nucleotides, at least about 7 nucleotides, at least about 9 nucleotides, at least about 11 nucleotides, or at least about 17 nucleotides. The fragment is typically less than about 300 nucleotides, less than about 100 nucleotides, less than about 75 nucleotides, less than about 50 nucleotides, or less than 30 nucleotides. In certain embodiments, the fragments can be used in polymerase chain reaction (PCR), various hybridization procedures or microarray procedures to identify or amplify identical or related parts of mRNA or DNA molecules. A fragment or segment may uniquely identify each polynucleotide sequence of the present invention.

“Genomic nucleic acid” or “genomic DNA” refers to some or all of the DNA from a chromosome. Genomic DNA may be intact or fragmented (e.g., digested with restriction endonucleases by methods known in the art). In some embodiments, genomic DNA may include sequence from all or a portion of a single gene or from multiple genes. In contrast, the term “total genomic nucleic acid” is used herein to refer to the full complement of DNA contained in the genome. Methods of purifying DNA and/or RNA from a variety of samples are well-known in the art.

As used herein, the term “oligonucleotide” refers to a short polymer composed of deoxyribonucleotides, ribonucleotides or any combination thereof. Oligonucleotides are generally at least about 10, 11, 12, 13, 14, 15, 20, 25, 40 or 50 up to about 100, 110, 150 or 200 nucleotides (nt) in length, such as from about 10, 11, 12, 13, 14, or 15 up to about 70 or 85 nt, such as from about 18 up to about 26 nt in length. The single letter code for nucleotides is as described in the U.S. Patent Office Manual of Patent Examining Procedure, section 2422, table 1. In this regard, the nucleotide designation “R” means purine such as guanine or adenine, “Y” means pyrimidine such as cytosine or thymidine (uracil if RNA); and “M” means adenine or cytosine. An oligonucleotide may be used as a primer or as a probe.

As used herein, a “primer” for amplification is an oligonucleotide that is complementary to a target nucleotide sequence and leads to addition of nucleotides to the 3′ end of the primer in the presence of a DNA or RNA polymerase. The 3′ nucleotide of the primer should generally be identical to the target nucleic acid sequence at a corresponding nucleotide position for optimal expression and amplification. The term “primer” as used herein includes all forms of primers that may be synthesized including peptide nucleic acid primers, locked nucleic acid primers, phosphorothioate modified primers, labeled primers, and the like. As used herein, a “forward primer” is a primer that is complementary to the anti-sense strand of dsDNA. A “reverse primer” is complementary to the sense-strand of dsDNA. An “exogenous primer” refers specifically to an oligonucleotide that is added to a reaction vessel containing the sample nucleic acid to be amplified from outside the vessel and is not produced from amplification in the reaction vessel. A primer that is “associated with” a fluorophore or other label is connected to the label through some means. An example is a primer-probe.

Primers are typically from at least 10, 15, 18, or 30 nucleotides in length up to about 100, 110, 125, or 200 nucleotides in length, such as from at least 15 up to about 60 nucleotides in length, and such as from at least 25 up to about 40 nucleotides in length. In some embodiments, primers and/or probes are 15 to 35 nucleotides in length. There is no standard length for optimal hybridization or polymerase chain reaction amplification. An optimal length for a particular primer application may be readily determined in the manner described in H. Erlich, PCR Technology, Principles and Application for DNA Amplification, (1989).

A “primer pair” is a pair of primers that are both directed to target nucleic acid sequence. A primer pair contains a forward primer and a reverse primer, each of which hybridizes under stringent condition to a different strand of a double-stranded target nucleic acid sequence. The forward primer is complementary to the anti-sense strand of the dsDNA and the reverse primer is complementary to the sense-strand. One primer of a primer pair may be a primer-probe (i.e., a bi-functional molecule that contains a PCR primer element covalently linked by a polymerase-blocking group to a probe element and, in addition, may contain a fluorophore that interacts with a quencher).

An oligonucleotide (e.g., a probe or a primer) that is specific for a target nucleic acid will “hybridize” to the target nucleic acid under specified conditions. As used herein, “hybridization” or “hybridizing” refers to the process by which an oligonucleotide single strand anneals with a complementary strand through base pairing under defined hybridization conditions.

“Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tm) for the specific sequence at a defined ionic strength and pH. The Tm is the temperature (under defined ionic strength and pH) at which 50% of the target nucleic acid hybridizes to a perfectly matched probe. Equations for calculating Tm and conditions for nucleic acid hybridization are known in the art. Specific hybridization may occur under stringent conditions, which are well known in the art. Stringent hybridization conditions are hybridization in 50% formamide, 1 M NaCl, 1% SDS at 37° C., and a wash in 0.1×SSC at 60° C. Hybridization procedures are well known in the art and are described in e.g. Ausubel et al, Current Protocols in Molecular Biology, John Wiley & Sons Inc., 1994.

As used herein, an oligonucleotide is “specific” for a nucleic acid if the oligonucleotide has at least 50% sequence identity with the nucleic acid when the oligonucleotide and the nucleic acid are aligned. An oligonucleotide that is specific for a nucleic acid is one that, under the appropriate hybridization or washing conditions, is capable of hybridizing to the target of interest and not substantially hybridizing to nucleic acids which are not of interest. In some embodiments, higher levels of sequence identity include at least 75%, at least 80%, at least 85%, at least 90%, at least 95% and at least 98% sequence identity. Sequence identity can be determined using a commercially available computer program with a default setting that employs algorithms well known in the art. As used herein, sequences that have “high sequence identity” have identical nucleotides at least at about 50% of aligned nucleotide positions, such as at least at about 60% of aligned nucleotide positions, such as at least at about 75% of aligned nucleotide positions.

Oligonucleotides used as primers or probes for specifically amplifying (i.e., amplifying a particular target nucleic acid) or specifically detecting (i.e., detecting a particular target nucleic acid sequence) a target nucleic acid generally are capable of specifically hybridizing to the target nucleic acid under stringent conditions.

As used herein, the term “sample” or “test sample” may comprise clinical samples, isolated nucleic acids, or isolated microorganisms. In some embodiments, a sample is obtained from a biological source (i.e., a “biological sample”), such as tissue, bodily fluid, or microorganisms collected from a subject. Sample sources include, but are not limited to, sputum (processed or unprocessed), bronchial alveolar lavage (BAL), bronchial wash (BW), blood, bodily fluids, cerebrospinal fluid (CSF), urine, plasma, serum, or tissue (e.g., biopsy material). Exemplary sample sources include nasopharyngeal swabs, wound swabs, and nasal washes. The term “patient sample” as used herein refers to a sample obtained from a human seeking diagnosis and/or treatment of a disease.

As used herein, the term “polymorphism” refers to the existence of two or more different nucleotide sequences at a particular locus in the DNA of the genome. Polymorphisms can serve as genetic markers and may also be referred to as genetic variants. Polymorphisms include nucleotide substitutions, insertions, deletions and microsatellites, and may, but need not, result in detectable differences in gene expression or protein function. A polymorphic site is a nucleotide position within a locus at which the nucleotide sequence varies from a reference sequence in at least one individual in a population.

A “variant” or “genetic variant” as used herein, refers to a specific isoform of a haplotype found in a population, the specific form differing from other forms of the same haplotype in at least one, and frequently more than one, variant sites or nucleotides within the region of interest in the gene. The sequences at these variant sites that differ between different alleles of a gene are termed “gene sequence variants,” “alleles,” or “variants.” The term “alternative form” refers to an allele that can be distinguished from other alleles by having at least one, and frequently more than one, variant sites within the gene sequence. “Variants” include isoforms having single nucleotide polymorphisms (SNPs) and deletion/insertion polymorphisms (DIPs). Reference to the presence of a variant means a particular variant, i.e., particular nucleotides at particular polymorphic sites, rather than just the presence of any variance in the gene.

The term “genotype”, as used herein, refers to the particular allelic form of a gene, which can be defined by the particular nucleotide(s) present in a nucleic acid sequence at a particular site(s). Genotype may also indicate the pair of alleles present at one or more polymorphic loci. For diploid organisms, such as humans, two haplotypes make up a genotype. Genotyping is any process for determining a genotype of an individual, e.g., by nucleic acid amplification, DNA sequencing, antibody binding, or other chemical analysis (e.g., to determine the length). The resulting genotype may be unphased, meaning that the sequences found are not known to be derived from one parental chromosome or the other.

As used herein, “expression of a genomic locus” or “gene expression” is the process by which information from a gene is used in the synthesis of a functional gene product. The products of gene expression are often proteins, but in non-protein coding genes such as IRNA genes or tRNA genes, the product is functional RNA. The process of gene expression is used by all known life—eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context.

As used herein, the term “modulation” or “modulating” in the context of gene expression or function refers to effecting a change in the activity of a gene or a gene product therefrom. Modulation of expression can include, but is not limited to, gene activation and gene repression. Modulation of expression of a gene can refer to reducing or increasing the expression of a gene. Genome editing (e.g., cleavage, alteration, inactivation, random mutation) can be used to modulate expression of a gene. Gene inactivation refers to any reduction in gene expression as compared to a cell that does not include a gene modulation system as described herein. Thus, gene inactivation may be partial or complete.

As used herein, the term “detecting” refers to observing a signal from a detectable label to indicate the presence of a target. More specifically, detecting is used in the context of detecting a specific sequence of a target nucleic acid molecule. The term “detecting” used in context of detecting a signal from a detectable label to indicate the presence of a target nucleic acid in the sample does not require the method to provide 100% sensitivity and/or 100% specificity. In some embodiments, sensitivity is at least 50%, at least 60%, at least 70%, at least 80%, at least 90%, or at least 99%. In some embodiments, the specificity is at least 50%, at least 60%, at least 70%, at least 80%, at least 90%, or at least 99%. Detecting also encompasses assays that produce false positives and false negatives. False negative rates can be 1%, 5%, 10%, 15%, 20% or even higher. False positive rates can be 1%, 5%, 10%, 15%, 20% or even higher. As used herein, “detecting” may also refer to observing a signal indicating the presence and/or amount of a substance, such as a protein in a sample. “Detection” includes any means for detection, including direct and indirect detection. In some embodiments, expression may be detected and measured using one or more methods. For example, protein expression may be analyzed by any suitable process for detecting protein, such as immunohistochemistry, microarray, western blot, mass-spectrometry, ELIZA, or other enzyme activity assay. Nucleic acid detection may be performed using any suitable process for detecting a nucleic acid, such as sequence, southern blot, PCR, CHIP-seq, probe oligonucleotides, microarray, or hybrid capture.

As used herein, the term “effector protein” refers to a protein that modulates a biological function of a cell when introduced into the cell, e.g., a modification of a nucleic acid molecule in the cell (such as a cleavage, deamination, recombination, etc.), or a modulation (e.g., increases or decreases) the expression or the expression level of a gene in the cell. In some embodiments, the effector protein comprises a zinc finger nuclease, a TALEN, a Cas protein, Cpf1 (Cas12), C2c2 (Cas13), Csm/Cmr, C2c6, Csy4, Csm6, C2c2c, dC2c, or Fanzor. In some embodiments, the effector protein comprises endonuclease-, nickase-, or nuclease-dead embodiments of TALEN, Cas protein, Cpf1 (Cas12), C2c2 (Cas13), Csm/Cmr, C2c6, Csy4, Csm6, C2c2c, dC2c, or Fanzor.

As used herein, the term “CRISPR/Cas” or “clustered regularly interspaced short palindromic repeats” or “CRISPR” refers to DNA loci containing short repetitions of base sequences followed by short segments of spacer DNA from previous exposures to a virus or plasmid. Bacteria and archaea have evolved adaptive immune defenses termed CRISPR/CRISPR-associated (Cas) systems that use short RNA to direct degradation of foreign nucleic acids. In bacteria, the CRISPR system provides acquired immunity against invading foreign DNA via RNA-guided DNA cleavage.

As used herein, the term “CRISPR/Cas9” system or “CRISPR/Cas9-mediated gene editing” refers to a type II CRISPR/Cas system that has been modified for genome editing/engineering. It is typically comprised of a “guide” RNA (gRNA) and a non-specific CRISPR-associated endonuclease (Cas9).

As used herein, the term “guide” is used interchangeably with “guide RNA (gRNA)”, “short guide RNA (sgRNA)”, or “single guide RNA (sgRNA). The sgRNA is a short synthetic RNA composed of a “scaffold” sequence necessary for Cas9-binding and a user-defined ~20 nucleotide “spacer” or “targeting” sequence which defines the genomic target to be modified. The genomic target of Cas9 can be changed by changing the targeting sequence present in the sgRNA.

As used herein, the term “CRISPRi” system refers to a modification of the CRISPR-Cas9 system that functions to repress or decrease gene expression. In certain embodiments, the CRISPRi system is comprised of a catalytically dead RNA/DNA guided endonuclease, such as dCas9, dCas12a/dCpf1, dCas12b/dC2c1, dCas12c/dC2c3, dCas12d/dCasY, dCas12e/dCasX, dCas13a/dC2c2, dCas13b, dCas13c, dCas14, dead Cascade complex, or others; at least one transcriptional repressor; and at least one sgRNA that functions to repress expression of at least one gene of interest. The term “repression” as used herein refers to a decrease in gene expression of one or more genes.

As used herein, the term “CRISPRa” system refers to a modification of the CRISPR-Cas9 system that functions to activate or increase gene expression. In certain embodiments, the CRISPRa system is comprised of a catalytically dead RNA/DNA guided endonuclease, such as dCas9, dCas12a/dCpf1, dCas12b/dC2c1, dCas12c/dC2c3, dCas12d/dCasY, dCas12e/dCasX, dCas13a/dC2c2, dCas13b, dCas13c, dCas14, dead Cascade complex, or others; at least one transcriptional activator; and at least one sgRNA that functions to increase expression of at least one gene of interest. The term “activation” as used herein refers to an increase in gene expression of one or more genes.

As used herein, the term “dCas9” as used herein refers to a catalytically dead Cas9 protein that lacks endonuclease activity. “dSaCas9” refers to dCas9 derived from Staphylococcus aureus. dSpCas9″ refers to dCas9 derived from Streptococcus pyogenes.

As used herein, “expression” or “expressing” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. As used herein “expression” of a gene or nucleic acid encompasses not only cellular gene expression, but also the transcription and translation of nucleic acid(s) in cloning systems and in any other context.

The term “downregulation” as used herein refers to the decrease or elimination of gene expression of one or more genes.

The term “knockdown” as used herein refers to a decrease in gene expression of one or more genes.

The term “knockout” as used herein refers to the ablation of gene expression of one or more genes.

The term “protospacer adjacent motif (or PAM) as used herein, refers to a DNA sequence that may be required for a Cas9/sgRNA to form an R-loop to interrogate a specific DNA sequence through Watson-Crick pairing of its guide RNA with the genome. The PAM specificity may be a function of the DNA-binding specifi city of the Cas9 protein (e.g., a “protospacer adjacent motif recognition domain” at the C-terminus of Cas9).

As used herein, the term “sgRNA” refers to single guide RNA used in conjunction with CRISPR associated systems (Cas). sgRNAs are a fusion of crRNA and tracrRNA and contain nucleotides of sequence complementary to the desired target site. Watson-Crick pairing of the sgRNA with the target site permits R-loop formation, which in conjunction with a functional PAM permits DNA cleavage or in the case of nuclease-deficient Cas9 allows tight binding to the DNA at that locus.

As used herein, the term “guide sequence” refers to the about 20 bp sequence within the guide RNA that specifies the target site and may be used interchangeably with the terms “guide” or “spacer”. The term “guide sequence” herein also includes the corresponding DNA or DNA encoding the RNA guide sequence.

As used herein, the term “target sequence” refers to the sequence to which the guide binds. In some embodiments, the target sequence may comprise “repeated genomic loci” or “repeated nucleic acid sequences”, which are sequences found in two or more target regions. In some embodiments, the repeated nucleic acid sequences are found in in non-coding regulatory elements, such as promoters, enhancers, transposable elements, 5′UTRs, or introns, and transcription factor binding motifs.

As used herein, the term “transcription factor” or “TF” refers generally to proteins that are involved in gene regulation in both prokaryotic and eukaryotic organisms. Generally, transcription factors bind to a consensus sequence, or transcription factor binding sequence to elicit an effect. In one embodiment, transcription factors can have a positive effect on gene expression and, thus, may be referred to as an “activator” or a “transcriptional activation factor.” In another embodiment, a transcription factor can negatively affect gene expression and, thus, may be referred to as “repressors” or a “transcription repression factor.” Activators and repressors are generally used terms and their functions are discerned by those skilled in the art. In some embodiments, the transcription factors and transcription factor binding sequences used in the methods described herein include motifs contain a “GG” or “CC” (for the reverse complement) sequence, making them amenable to dCas9 (NGG PAM) targeting. In some embodiments, the transcription factors described herein are members of the Ets tryptophan cluster factors family of transcription factors, the C2H2 zinc finger factors family or transcription factors, or the nuclear receptors with C4 zinc fingers family of transcription factors. In some embodiments, the transcription factors and transcription factor binding sequences used in the methods described herein include PU.1, SP1, YY1, or NR2. Non-limiting examples of the transcription factors and transcription factor binding sequences used in the methods described herein include AhR:Arnt, AIRE, alpha-CP1, Alx-4, AML AP-2, AP-2alphaA, AR, AREB6, ATF6, BCL6, Brachyury, CACCC-binding factor, CDP, c-Ets-1 p54, c-Myc, COUP direct repeat 1, CP2/LBP-1c/LSF, c-Rel, CCCTC-binding factor (CTCF), CTF1, deltaEF1, E12, E2F-1, E47, EBF, Egr-1, Egr-3, Elf-1, Elk-1, ER, Ets, ETS2, FXR inverted repeat 1, FXR/RXR-alpha, GABP, GAF, GCM, GLI, GLI1, Hand1:E47, HEB, Helios A, HEN1, HNF4 direct repeat 1, HNF4, COUP, Ikaros, KAISO, LM4_M2, LRF, LRH1, LUN-1, LXR, LXR direct repeat 4, LXR, PXR, CAR, COUP, RAR, Lyf-1, MAF, MIF-1, MIZF, MyoD, myogenin/NF-1, NERF1a, NF-1, NF-AT, NF-kappaB, NF-kappaB (p50), NF-kappaB (p65), NF-muE1, NF-Y, NGFI-C, NRSF, NUR77, Olf-1, P50: RELA-P65, p53, Pax, Pax-1, Pax-6, PEBP, Pitx2, PPAR, PPAR direct repeat 1, PPARalpha:RXRalpha, PPARgamma:RXRalpha, PPARgamma, PU.1, PXR, CAR, LXR, FXR, RBP-Jkappa, RelB:p52 (NF-kappaB), REST, RFX1, Roaz, RORalpha, RORalpha1, RORalpha2, RP58, SAP-1a, SF1, Sp3, SREBP, SREBP-1, SRF, Staf, STAT1, STAT3, STAT5A (homodimer), STAT5B (homodimer), STATx, SZF1-1, TAL1, Tax/CREB, TBX22, TBX5, Tel-2, VDR, YY1, ZBRK1, Zic2, and ZID.

### III. CRISPR for Gene Modulation and Regulatory Element Analysis

CRISPR interference (CRISPRi) and CRISPR activation (CRISPRa) consists of a catalytically dead Cas9 (dCas9) that can be fused to an effector domain, such as a transposase domain, integrase domain, recombinase domain, resolvase domain, invertase domain, protease domain, DNA methyltransferase domain, DNA hydroxylmethylase domain, DNA demethylase domain, histone acetylase domain, histone deacetylases domain, nuclease domain, repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase, or histone tail protease. Repression domains, without limitation, include KRAB, DNMT1, HDAC, DNMT3A, DNMT3A/3L, MeCP2, SID, NuRD, HP1, LSD1, SETDB1, EZH2, or a combination thereof. Activator domains, without limitation, include VP64, p65, VPR (VP64-p65-Rta), p65-HSF1, VPR-RecA, or a combination thereof. The non-limiting examples described herein utilize the domain zinc-finger repressive protein (KRAB), hereafter referred to as CRISPRi. Studies have generally relied on CRISPRi-directed targeting of CREs followed by RNA measurement or flow cytometry to detect gene expression changes (Canver et al (2015) Nature, 257(7577):192-197; Chen et al. (2023) Cell Genomics, 3(6):100318; Fulco et al. (2016) Science, 354(6313):769-773); Gasperini et al. (2019) Cell, 176(6):1516; Gilbert et al. (2023) Cell, 154(2):442-451; Korkmaz et al. (2016) Nature Biotechnology, 37(6):657-666; Nasser et al. (2021) Nature, 593(7858):238-243; Reilly et al. (2021) Nature Genetics, 53(8):1166-1176; Sanjana et al. (2016) Science, 353(6307):1545-1549; Thakore et al. (2015) Nature Methods, 12(12):1143-1149). However, efforts to characterize CREs at scale have been complicated by the multitude of putative elements and by mild effect sizes. High multiplicity of infection (MOI) delivery of guides paired with single-cell RNAseq provided a multiplexed testing approach (Gasperini et al. (2019) Cell, 176(6):1516), though at the cost of many viral integration events. The CRISPRi-based approaches described herein can effectively assess significant CREs and require a large scale.

The Cas9 nuclease is honed by a spacer sequence (sgRNA) that determines targeting specificity. Typically, spacers are 20 nucleotides (nt) in length and target a single genomic site. Early studies posited that spacers with minor 5′ truncations or mismatches retain Cas9-mediated, on-target cleavage (Fu et al. (2013) Nature Biotechnology, 31(9):822-826; Jinek et al. (2012), Science, 337(6096):816-821; Mali et al. (2013) Nature Biotechnology, 31(9):833-838). The 3′ end of the spacer sequence, also termed the seed sequence, is necessary though not alone sufficient for on-target cleavage. Activity was observed with truncated spacers of 17nt while 15nt or shorter spacers failed to demonstrate cleavage activity (Cencic et al. (2014) PLOS One, 9(10):e109213; Fu et al. (2014), Nature Biotechnology, 32(3):279-284; Jinek (2012); Zhang et al. (2016) Scientific Reports, 6(1)). However, an important distinction exists in the requirements for Cas9 binding and cleavage that is illuminated with dCas9 protein. Indeed, spacers as short as 10nt sufficed for dCas9-VPR (CRISPRa) activity at a single target site (Kiani et al. (2015) Nature Methods, 12(11):1051-1054). It was therefore postulated that KRAB-dCas9 (CRISPRi) would perform similarly and, further, target multiple intended sites simultaneously.

Here, the ability of truncated guides to direct CRISPRi to multiple sites simultaneously in the genome for multi-locus repression was examined. Truncated guides resulted in reliable on-target efficacy down to spacer lengths of 9nt. TF motifs, which are often less than 14nt, presented ideal genomic loci for multiplexed repression. TF motifs in a CRE of the EPB41 gene were targeted and comparable on-target efficiencies with full-length and truncated guides was observed. A truncated guide library targeting thousands of CTCF motif sites and discovered significant CTCF disruption was screened. The invention disclosed herein offers a new opportunity to simultaneously perturb CREs at scale and effectively prioritize genomic loci for further study.

### IV. Cells, Vectors, and Polynucleotides

In some aspects, the present disclosure provides an engineered, non-naturally occurring vector system comprising (a) one or more vectors comprising a first regulatory element operably linked to the present guide RNA (e.g., sgRNAs described herein) that targets a DNA molecule encoding a gene regulatory element and (b) a second regulatory element operably linked to an effector protein. Components (a) and (b) may be located on the same or different vectors of the system. The present guide RNA targets the DNA molecule encoding the gene product in a cell and the effector protein modifies the expression of the DNA molecule encoding the gene product; and, wherein the effector protein and the guide RNA do not naturally occur together.

In some aspects, the present disclosure provides a vector system comprising one or more vectors, wherein the one or more vectors comprises: (a) a first expression regulatory element operably linked to a nucleotide sequence encoding an effector protein, or one or more nucleotide sequences encoding the effector protein; and (b) a second expression regulatory element operably linked to one or more nucleotide sequences encoding a single guide RNA (sgRNA) comprising a guide sequence capable of hybridizing to a target sequence, wherein components (a) and (b) are located on same or different vectors. In some embodiments, the sgRNA is capable of hybridizing to one or more gene regulatory elements in a promotor or enhancer for example, (i.e., target sequences).

In some embodiments, guide RNA forms a complex with the effector protein to form an effector protein complex. For example, a guide RNA can form a complex with a CRISPR enzyme to form a CRISPR complex. The effector protein complex or polynucleotides encoding it may comprise one or more nuclear localization sequences of sufficient strength to drive accumulation of said effector protein complex in a detectable amount in the nucleus of a eukaryotic cell.

In some embodiments, the effector protein is a CRISPR enzyme. In some embodiments, the CRISPR enzyme is a Cas9 enzyme. In some embodiments, the Cas9 enzyme is S. aureus, S. pneumoniae, S. pyogenes, or S. thermophilus Cas9, and may include mutated Cas9 derived from these organisms. The enzyme may be a Cas9 homolog or ortholog. In some embodiments, the CRISPR enzyme is codon-optimized for expression in a eukaryotic cell. In some embodiments, the CRISPR enzyme directs cleavage of one or two strands at the location of the target sequence. In some embodiments, the effector protein is a CRISPR enzyme/repressor domain fusion protein. In some embodiments, the repression domain is KRAB, DNMT1, HDAC, DNMT3A, DNMT3A/3L, MeCP2, SID, NuRD, HP1, LSD1, SETDB1, EZH2, or a combination thereof.

In some embodiments, the first regulatory element in the present vectors is a polymerase III promoter. In some embodiments, the second regulatory element in the present vectors is a polymerase II promoter.

In some embodiments, the guide sequence is at least 8, 9, 10, 11, 12, 13, 14, 15, 16, 17, 18, 19, 20, 25 nucleotides, or between 9-21, or between 9-14, or between 9-11 nucleotides in length.

In any of the sgRNA, DNA polynucleotide molecule, DNA expression vector, delivery vector, method, system, composition or complex described herein said manipulation may be performed in vitro or ex vivo.

It will be appreciated that the invention described herein involves various components which may display variations in their specific characteristics. It will be appreciated that any combination of features described above and herein, as appropriate, are contemplated as a means for implementing the invention.

In general, applying to any of the aspects discussed herein, the sgRNA is a non-naturally occurring single guide RNA molecule. It is capable of effecting the manipulation of a target nucleic acid within a prokaryotic or eukaryotic cell when in complex within the cell with an effector protein (e.g., a CRISPR enzyme). Examples of CRISPR enzymes are a Staphylococcus aureus Cas9 enzyme (SaCas9) or other similar smaller Cas9 orthologs. The sgRNA may comprise, in some embodiments, the following, in any tandem arrangement:
I. a guide sequence, which is capable of hybridizing to a sequence of the target nucleic acid to be manipulated;
II. a tracr mate sequence, comprising a region of sense sequence;
III. a linker sequence; and
IV. a tracr sequence, comprising a region of antisense sequence which is positioned adjacent the linker sequence and which is capable of hybridizing with the region of sense sequence thereby forming a stem loop.

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

The term “regulatory element” is intended to include promoters, enhancers, internal ribosomal entry sites (IRES), and other expression control elements (e.g., transcription termination signals, such as polyadenylation signals and poly-U sequences). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol I promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Advantageous vectors include lentiviruses and adeno-associated viruses (AAV), and types of such vectors can also be selected for targeting particular types of cells.

In one aspect, the invention provides a eukaryotic host cell. The host cell may comprise (a) a first expression regulatory element operably linked to a nucleotide sequence encoding an effector protein, or one or more nucleotide sequences encoding the effector protein; and (b) a second expression regulatory element operably linked to one or more nucleotide sequences encoding a single guide RNA (sgRNA) comprising a guide sequence capable of hybridizing to a target sequence. In some embodiments, the host cell comprises components (a) and (b). In some embodiments, component (a), component (b), or components (a) and (b) are stably integrated into a genome of the host eukaryotic cell. In some embodiments, component (a) further comprises the tracr sequence downstream of the tracr mate sequence under the control of the first regulatory element. In some embodiments, component (b) further comprises two or more guide sequences operably linked to the first regulatory element, wherein when expressed, each of the two or more guide sequences direct sequence specific binding of an effector protein complex (e.g., a CRISPR complex) to a different target sequence in a eukaryotic cell. The effector protein may be an enzyme, such as a CRISPR enzyme (e.g., a Cas9 homolog or ortholog). In some embodiments, the CRISPR enzyme is codon-optimized for expression in a eukaryotic cell. In some embodiments, the CRISPR enzyme directs cleavage of one or two strands at the location of the target sequence. In some embodiments, the CRISPR enzyme lacks DNA strand cleavage activity. In some embodiments, the effector protein is a CRISPR enzyme/repressor domain fusion protein. In some embodiments, the repression domain is KRAB, DNMT1, HDAC, DNMT3A, DNMT3A/3L, MeCP2, SID, NuRD, HP1, LSD1, SETDB1, EZH2, or a combination thereof. In some embodiments, the guide sequence is at least 8, 9, 10, 11, 12, 13, 14, 15, 16, 17, 18, 19, 20, 25 nucleotides, or between 9-21, or between 9-14, or between 9-11 nucleotides in length. In one aspect, the invention provides a non-human eukaryotic organism, such as a multicellular eukaryotic organism, comprising a eukaryotic host cell according to any of the described embodiments. In other aspects, the invention provides a eukaryotic organism, such as multicellular eukaryotic organism, comprising a eukaryotic host cell according to any of the described embodiments. The organism in some embodiments of these aspects may be an animal, for example a mammal.

With respect to use of the CRISPR-Cas system generally, mention is made of the documents, including patent applications, patents, and patent publications cited throughout this disclosure as embodiments of the invention can be used as in those documents. For example, WO 2014/093622 (PCT/US2013/074667), incorporated herein by reference.

In one aspect, the present disclosure provides a vector system or eukaryotic host cell comprising (a) a first expression regulatory element operably linked to a nucleotide sequence encoding an effector protein, or one or more nucleotide sequences encoding the effector protein; and (b) a second expression regulatory element operably linked to one or more nucleotide sequences encoding a single guide RNA (sgRNA) comprising a guide sequence capable of hybridizing to a target sequence. Components (a) and (b) can be on the same or different vectors. In some embodiments, the sgRNA is capable of hybridizing to one or more TF binding sequences. In some embodiments, the host cell comprises components (a) and (b). In some embodiments, component (a), component (b), or components (a) and (b) are stably integrated into a genome of the host eukaryotic cell.

### V. Kits

The present disclosure also contemplates detection, discovery, and analysis systems in kit form. In some embodiments, a kit can be used for conducting the detecting, discovering, or characterizing regulatory elements described herein.

The kit may contain, in a carrier or compartmentalized container, reagents useful in any of the above-described embodiments of the methods.

A detection system provided herein can include a kit that contains, in an amount sufficient for at least one assay, any of the hybridization assay probes and amplification primers for detection of nucleic acids encoding one or more TF binding sequences, such as those discussed herein.

In some embodiments, the kit includes one or more primers or probes suitable for amplification and/or sequencing. The primers can be labeled with a detectable marker such as radioactive isotopes, or fluorescence markers.

In some embodiments, the kit may also include primers for the amplification of one or more housekeeping genes. Non-limiting examples of housekeeping genes include GAPDH, ACTB, TUBB, UBQ, PGK, and RPL.

Typically, the kits will also include instructions recorded in a tangible form (e.g., contained on paper or an electronic medium) for using the packaged probes, primers, and/or antibodies in a detection assay for determining the presence or amount of mutant nucleic acid or protein in a test sample.

The various components of the detection, discovery, and analysis systems can be provided in a variety of forms. For example, the required enzymes, the nucleotide triphosphates, the probes, primers, and/or antibodies can be provided as a lyophilized reagent. These lyophilized reagents can be pre-mixed before lyophilization so that when reconstituted they form a complete mixture with the proper ratio of each of the components ready for use in the assay. In addition, the systems of the present inventions can contain a reconstitution reagent for reconstituting the lyophilized reagents of the kit. In an exemplary kit, the enzymes, nucleotide triphosphates and required cofactors for the enzymes are provided as a single lyophilized reagent that, when reconstituted, forms a proper reagent for use in the present amplification methods.

In some embodiments, the kit includes suitable buffers, reagents for isolating nucleic acid, and instructions for use. Kits can also include a microarray that contains nucleic acid or peptide probes for the detection of genes or encoded proteins, respectively.

In some embodiments, the kits can further contain a solid support for anchoring the nucleic acid or proteins of interest on the solid support. In some embodiments, the target nucleic acid can be anchored to the solid support directly or indirectly through a capture probe anchored to the solid support and capable of hybridizing to the nucleic acid of interest. Examples of such solid supports include, but are not limited to, beads, microparticles (for example, gold and other nanoparticles), microarray, microwells, multiwell plates. The solid surfaces can comprise a first member of a binding pair and the capture probe or the target nucleic acid can comprise a second member of the binding pair. Binding of the binding pair members will anchor the capture probe or the target nucleic acid to the solid surface. Examples of such binding pairs include but are not limited to biotin/streptavidin, hormone/receptor, ligand/receptor, antigen/antibody.

Exemplary packaging for the kit can include, for example, a container or support, in the form of, e.g., bag, box, tube, rack, and is optionally compartmentalized. The packaging can define an enclosed confinement for safety purposes during shipment and storage.

In one aspect, the present disclosure provides a kit comprising one or more of the components described herein. In some embodiments, the kit comprises a vector system and instructions for using the kit. In some embodiments, the vector system comprises (a) a first expression regulatory element operably linked to a nucleotide sequence encoding an effector protein, or one or more nucleotide sequences encoding the effector protein; and (b) a second expression regulatory element operably linked to one or more nucleotide sequences encoding a single guide RNA (sgRNA) comprising a guide sequence capable of hybridizing to a target sequence. In some embodiments, components (a) and (b) are located on the same or different vectors. In some embodiments, the sgRNA is capable of hybridizing to one or more TF binding sites. In some embodiments, the host cell comprises components (a) and (b). In some embodiments, component (a), component (b), or components (a) and (b) are stably integrated into a genome of the host eukaryotic cell. In some embodiments, the effector protein is a CRISPR enzyme. In some embodiments, the CRISPR enzyme is codon-optimized for expression in a eukaryotic cell. In some embodiments, the CRISPR enzyme directs cleavage of one or two strands at the location of the target sequence. In some embodiments, the CRISPR enzyme lacks DNA strand cleavage activity. In some embodiments, the effector protein is a CRISPR enzyme/repressor domain fusion protein. In some embodiments, the repression domain is KRAB, DNMT1, HDAC, DNMT3A, DNMT3A/3L, MeCP2, SID, NuRD, HP1, LSD1, SETDB1, EZH2, or a combination thereof. In some embodiments, the guide sequence is at least 8, 9, 10, 11, 12, 13, 14, 15, 16, 17, 18, 19, 20, 25 nucleotides, or between 9-21, or between 9-14, or between 9-11 nucleotides in length.

### EXAMPLES

### Materials and Methods

Cell Culture—Jurkat, K562, and MV4-11 cells were cultured in RPMI (Gibco, 61870127)+10% FBS (Sigma, F2442) supplemented with 1% penicillin/streptomycin (Gibco, 15140163). HEK293FT and A375 cells were cultured in GlutaMAX High Glucose DMEM (Gibco, 10569044)+10% FBS (Sigma, F2442) supplemented with 1% penicillin/streptomycin. Cells were tested monthly (negative) for mycoplasma contamination and maintained in a 37° C. humidity-controlled incubator with 5% CO₂.

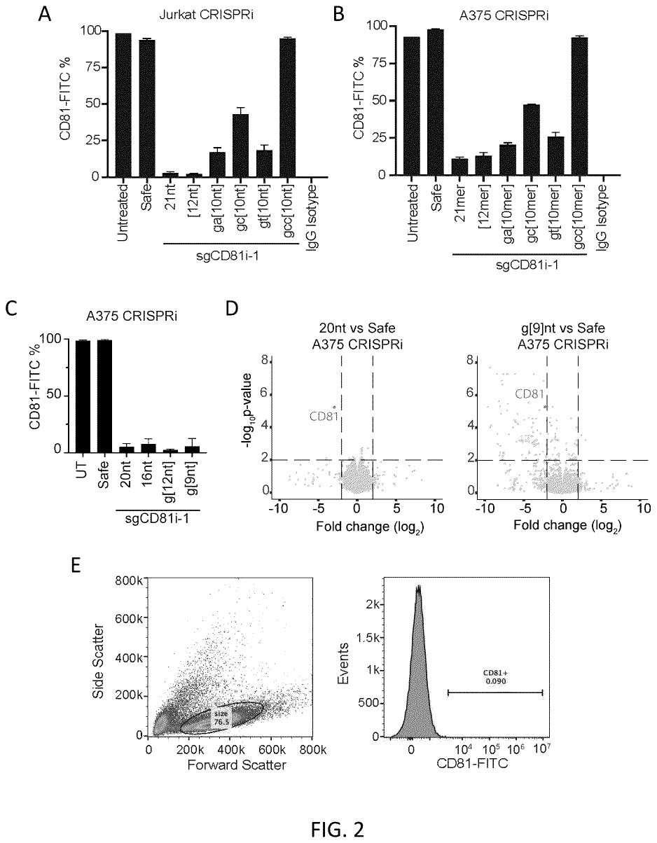

Flow Cytometry—Cells were incubated for 30 minutes at room temperature in 0.5% BSA PBS with 1:50 CD81-FITC antibody (Biolegend, 349504) or mouse IgG1 FITC isotype control antibody (Biolegend, 400107). Cells were washed twice prior to analysis on a Cytoflex (BD) cell analyzer. The gating strategy can be found in FIG. 2E.

Guide Selection—All guide sequences in this study can be found in Table 1 below. This includes the CTCF motif-directed library for the 24 guides (10nt, selected based on TF motif; SEQ ID NOs: 52-75) and 96 (11nt, by each base to the 5′ end of each 10nt guide; SEQ ID NOs: 77-172). To assess the effect of CTCF knockdown on cell fitness, CRISPick (Broad Institute) was used to select 20 sgRNAs targeting the CTCF promoter (SEQ ID NOs: 173-192). After screening, one guide was selected based on lethality across all cell lines and was included as the knockdown data found in Table 3. Additionally, 16 non-targeting controls were randomly selected from the Brunello library (Doench et al. (2016) Nature Biotechnology, 34(2):184-191) and included in the library with 15 Safe Harbor sgRNAs (Hess et al. (2016) Nature Methods, 13(12):1036-1042; SEQ ID NOs: 193-207) as negative controls.

TABLE 1
Guide sequences

| | | SEQ |
|---|---|---|
| Name | Guide Sequence | ID NO |
| Safe Harbor g[20 nt] | GCTAAAGTTGTCATTGATTT | 1 |
| sgCD81i-1 21 nt | GGCCTGGCAGGATGCGCGGTG | 2 |
| sgCD81i-1 20 nt | GCCTGGCAGGATGCGCGGTG | 3 |
| sgCD81i-1 g[18 nt] | gCTGGCAGGATGCGCGGTG | 4 |
| sgCD81i-1 g[17 nt] | gTGGCAGGATGCGCGGTG | 5 |
| sgCD81i-1 g[16 nt] | gGGCAGGATGCGCGGTG | 6 |
| sgCD81i-1 16 nt | GGCAGGATGCGCGGTG | 7 |
| sgCD81i-1 15 nt | GCAGGATGCGCGGTG | 8 |
| sgCD81i-1 g[13 nt] | gAGGATGCGCGGTG | 9 |
| sgCD81i-1 g[12 nt] | gGGATGCGCGGTG | 10 |
| sgCD81i-1 12 nt | GGATGCGCGGTG | 11 |
| sgCD81i-1 11 nt | GATGCGCGGTG | 12 |
| sgCD81i-1 g[9 nt] | TGCGCGGT | 13 |
| sgCD81i-1 g[8 nt] | GCGCGGTG | 14 |
| sgCD81i-2 20 nt | GCAGCCCTCCACTCCCATGG | 15 |
| sgCD81i-2 17 nt | GCCCTCCACTCCCATGG | 16 |
| sgCD81i-2 g[13 nt] | gTCCACTCCCATGG | 17 |
| sgCD81i-2 g[10 nt] | gACTCCCATGG | 18 |
| sgCD81i-3 g[20 nt] | gAGAGAGCGAGCGCGCAACGG | 19 |
| sgCD81i-3 17 nt | GAGCGAGCGCGCAACGG | 20 |
| sgCD81i-3 g[13 nt] | GAGCGCGCAACGG | 21 |
| sgCD81i-3 11 nt | GCGCGCAACGG | 22 |
| sgCD81-KO-1 g[20 nt] | gGTTGACAAAGCCCCAGATGC | 23 |
| sgCD81-KO-1 20 nt | GTTGACAAAGCCCCAGATGC | 24 |
| sgCD81-KO-1 g[18 nt] | gTGACAAAGCCCCAGATGC | 25 |
| sgCD81-KO-1 g[17 nt] | gGACAAAGCCCCAGATGC | 26 |
| sgCD81-KO-1 17 nt | GACAAAGCCCCAGATGC | 27 |
| sgCD81-KO-1 g[15 nt] | gCAAAGCCCCAGATGC | 28 |
| sgCD81-KO-2 g[20 nt] | gGCGCCCAACACCTTCTATGT | 29 |
| sgCD81-KO-2 20 nt | GCGCCCAACACCTTCTATGT | 30 |
| sgCD81-KO-2 g[18 nt] | gGCCCAACACCTTCTATGT | 31 |
| sgCD81-KO-2 18 nt | GCCCAACACCTTCTATGT | 32 |
| sgCD81-KO-2 g[16 nt] | gCCAACACCTTCTATGT | 33 |
| sgCD81-KO-2 g[15 nt] | gCAACACCTTCTATGT | 34 |
| EPB41_locus_PU.1 g[20 nt] | gAAAAGTTGAGACTGACAAAG | 35 |
| EPB41_locus_PU.1 g[13 nt] | gGAGACTGACAAAG | 36 |
| EPB41_locus_PU.1 11 nt | GACTGACAAAG | 37 |
| EPB41_locus_SP1 21 nt | GGAACAGACAGGTTGGGGGCG | 38 |
| EPB41_locus_SP1 g[13 nt] | gCAGGTTGGGGGCG | 39 |
| EPB41_locus_SP1 11 nt | GGTTGGGGGCG | 40 |
| EPB41_locus_YY1 21 nt | GCTTGAGGTGGGTGATAAAGA | 41 |
| EPB41_locus_YY1 14 nt | GTGGGTGATAAAGA | 42 |
| EPB41_locus_YY1 11 nt | GGTGATAAAGA | 43 |
| EPB41_locus_NR2 g[20 nt] | gATAGAGGGTGTGAAGGTGAC | 44 |
| EPB41_locus_NR2 14 nt | GGTGTGAAGGTGAC | 45 |
| EPB41_locus_NR2 11 nt | GTGAAGGTGAC | 46 |
| EPB41_validation_enh1 g[20 nt] | gGCTTCAGATGGGCTTGAGGT | 47 |
| EPB41_validation_enh2 21 nt | gACAGGATAAGGAATTCCTGG | 48 |
| EPB41_validation_enh3 g[20 nt] | gGGAGGTCCAGGCCTTCTCAG | 49 |
| EPB41_ promoter 21 nt | GATTCTGGAATTCGGGACCGC | 50 |
| CTCF_10 nt_sg1 | gTGCCCCCTAC | 52 |
| CTCF_10 nt_sg2 | gTGCCCTCTAC | 53 |
| CTCF_10 nt_sg3 | gTGCCCCCTTC | 54 |
| CTCF_10 nt_sg4 | gTGCCATCTAC | 55 |
| CTCF_10 nt_sg5 | gTGCCCCCTTG | 56 |
| CTCF_10 nt_sg6 | gTGCCCCCTAG | 57 |
| CTCF_10 nt_sg7 | gTGCCACCTAC | 58 |
| CTCF_10 nt_sg8 | gTGCCACCTAG | 59 |
| CTCF_10 nt_sg9 | gTGCCATCTTC | 60 |
| CTCF_10 nt_sg10 | gTGCCCTCTTG | 61 |
| CTCF_10 nt_sg11 | gTGCCACCTTC | 62 |
| CTCF_10 nt_sg12 | gTGCCCTCTTC | 63 |
| CTCF_10 nt_sg13 | gTGCCATCTGG | 64 |
| CTCF_10 nt_sg14 | gTGCCATCTAG | 65 |
| CTCF_10 nt_sg15 | gTGCCATCTTG | 66 |
| CTCF_10 nt_sg16 | gTGCCCTCTAG | 67 |
| CTCF_10 nt_sg17 | gTGCCACCTTG | 68 |
| CTCF_10 nt_sg18 | gTGCCCCCTGC | 69 |
| CTCF_10 nt_sg19 | gTGCCACCTGG | 70 |
| CTCF_10 nt_sg20 | gTGCCATCTGC | 71 |
| CTCF_10 nt_sg21 | gTGCCCCCTGG | 72 |
| CTCF_10 nt_sg22 | gTGCCCTCTGG | 73 |
| CTCF_10 nt_sg23 | gTGCCACCTGC | 74 |
| CTCF_10 nt_sg24 | gTGCCCTCTGC | 75 |
| CTCF_gaag[sg4] | gaagTGCCATCTAC | 76 |
| CTCF_1_10 nt + A | gATGCCCCCTAC | 77 |
| CTCF_1_10 nt + T | gTTGCCCCCTAC | 78 |
| CTCF_1_10 nt + G | gGTGCCCCCTAC | 79 |
| CTCF_1_10 nt + C | gCTGCCCCCTAC | 80 |
| CTCF_2_10 nt + A | gATGCCCTCTAC | 81 |
| CTCF_2_10 nt + T | gTTGCCCTCTAC | 82 |
| CTCF_2_10 nt + G | gGTGCCCTCTAC | 83 |
| CTCF_2_10 nt + C | gCTGCCCTCTAC | 84 |
| CTCF_3_10 nt + A | gATGCCCCCTTC | 85 |
| CTCF_3_10 nt + T | gTTGCCCCCTTC | 86 |
| CTCF_3_10 nt + G | gGTGCCCCCTTC | 87 |
| CTCF_3_10 nt + C | gCTGCCCCCTTC | 88 |
| CTCF_4_10 nt + A | gATGCCATCTAC | 89 |
| CTCF_4_10 nt + T | gTTGCCATCTAC | 90 |
| CTCF_4_10 nt + G | gGTGCCATCTAC | 91 |
| CTCF_4_10 nt + C | gCTGCCATCTAC | 92 |
| CTCF_5_10 nt + A | gATGCCCCCTTG | 93 |
| CTCF_5_10 nt + T | gTTGCCCCCTTG | 94 |
| CTCF_5_10 nt + G | gGTGCCCCCTTG | 95 |
| CTCF_5_10 nt + C | gCTGCCCCCTTG | 96 |
| CTCF_6_10 nt + A | gATGCCCCCTAG | 97 |
| CTCF_6_10 nt + T | gTTGCCCCCTAG | 98 |
| CTCF_6_10 nt + G | gGTGCCCCCTAG | 99 |
| CTCF_6_10 nt + C | gCTGCCCCCTAG | 100 |
| CTCF_7_10 nt + A | gATGCCACCTAC | 101 |
| CTCF_7_10 nt + T | gTTGCCACCTAC | 102 |
| CTCF_7_10 nt + G | gGTGCCACCTAC | 103 |
| CTCF_7_10 nt + C | gCTGCCACCTAC | 104 |
| CTCF_8_10 nt + A | gATGCCACCTAG | 105 |
| CTCF_8_10 nt + T | gTTGCCACCTAG | 106 |
| CTCF_8_10 nt + G | gGTGCCACCTAG | 107 |
| CTCF_8_10 nt + C | gCTGCCACCTAG | 108 |
| CTCF_9_10 nt + A | gATGCCATCTTC | 109 |
| CTCF_9_10 nt + T | gTTGCCATCTTC | 110 |
| CTCF_9_10 nt + G | gGTGCCATCTTC | 111 |
| CTCF_9_10 nt + C | gCTGCCATCTTC | 112 |
| CTCF_10_10 nt + A | gATGCCCTCTTG | 113 |
| CTCF_10_10 nt + T | gTTGCCCTCTTG | 114 |
| CTCF_10_10 nt + G | gGTGCCCTCTTG | 115 |
| CTCF_10_10 nt + C | gCTGCCCTCTTG | 116 |
| CTCF_11_10 nt + A | gATGCCACCTTC | 117 |
| CTCF_11_10 nt + T | gTTGCCACCTTC | 118 |
| CTCF_11_10 nt + G | gGTGCCACCTTC | 119 |
| CTCF_11_10 nt + C | gCTGCCACCTTC | 120 |
| CTCF_12_10 nt + A | gATGCCCTCTTC | 121 |
| CTCF_12_10 nt + T | gTTGCCCTCTTC | 122 |
| CTCF_12_10 nt + G | gGTGCCCTCTTC | 123 |
| CTCF_12_10 nt + C | gCTGCCCTCTTC | 124 |
| CTCF_13_10 nt + A | gATGCCATCTGG | 125 |
| CTCF_13_10 nt + T | gTTGCCATCTGG | 126 |
| CTCF_13_10 nt + G | gGTGCCATCTGG | 127 |
| CTCF_13_10 nt + C | gCTGCCATCTGG | 128 |
| CTCF_14_10 nt + A | gATGCCATCTAG | 129 |
| CTCF_14_10 nt + T | gTTGCCATCTAG | 130 |
| CTCF_14_10 nt + G | gGTGCCATCTAG | 131 |
| CTCF_14_10 nt + C | gCTGCCATCTAG | 132 |
| CTCF_15_10 nt + A | gATGCCATCTTG | 133 |
| CTCF_15_10 nt + T | gTTGCCATCTTG | 134 |
| CTCF_15_10 nt + G | gGTGCCATCTTG | 135 |
| CTCF_15_10 nt + C | gCTGCCATCTTG | 136 |
| CTCF_16_10 nt + A | gATGCCCTCTAG | 137 |
| CTCF_16_10 nt + T | gTTGCCCTCTAG | 138 |
| CTCF_16_10 nt + G | gGTGCCCTCTAG | 139 |
| CTCF_16_10 nt + C | gCTGCCCTCTAG | 140 |
| CTCF_17_10 nt + A | gATGCCACCTTG | 141 |
| CTCF_17_10 nt + T | gTTGCCACCTTG | 142 |
| CTCF_17_10 nt + G | gGTGCCACCTTG | 143 |
| CTCF_17_10 nt + C | gCTGCCACCTTG | 144 |
| CTCF_18_10 nt + A | gATGCCCCCTGC | 145 |
| CTCF_18_10 nt + T | gTTGCCCCCTGC | 146 |
| CTCF_18_10 nt + G | gGTGCCCCCTGC | 147 |
| CTCF_18_10 nt + C | gCTGCCCCCTGC | 148 |
| CTCF_19_10 nt + A | gATGCCACCTGG | 149 |
| CTCF_19_10 nt + T | gTTGCCACCTGG | 150 |
| CTCF_19_10 nt + G | gGTGCCACCTGG | 151 |
| CTCF_19_10 nt + C | gCTGCCACCTGG | 152 |
| CTCF_20_10 nt + A | gATGCCATCTGC | 153 |
| CTCF_20_10 nt + T | gTTGCCATCTGC | 154 |
| CTCF_20_10 nt + G | gGTGCCATCTGC | 155 |
| CTCF_20_10 nt + C | gCTGCCATCTGC | 156 |
| CTCF_21_10 nt + A | gATGCCCCCTGG | 157 |
| CTCF_21_10 nt + T | gTTGCCCCCTGG | 158 |
| CTCF_21_10 nt + G | gGTGCCCCCTGG | 159 |
| CTCF_21_10 nt + C | gCTGCCCCCTGG | 160 |
| CTCF_22_10 nt + A | gATGCCCTCTGG | 161 |
| CTCF_22_10 nt + T | gTTGCCCTCTGG | 162 |
| CTCF_22_10 nt + G | gGTGCCCTCTGG | 163 |
| CTCF_22_10 nt + C | gCTGCCCTCTGG | 164 |
| CTCF_23_10 nt + A | gATGCCACCTGC | 165 |
| CTCF_23_10 nt + T | gTTGCCACCTGC | 166 |
| CTCF_23_10 nt + G | gGTGCCACCTGC | 167 |
| CTCF_23_10 nt + C | gCTGCCACCTGC | 168 |
| CTCF_24_10 nt + A | gATGCCCTCTGC | 169 |
| CTCF_24_10 nt + T | gTTGCCCTCTGC | 170 |
| CTCF_24_10 nt + G | gGTGCCCTCTGC | 171 |
| CTCF_24_10 nt + C | gCTGCCCTCTGC | 172 |
| CTCF_promoter_1 | gCAGGCTCAGACACAAAATGG | 173 |
| CTCF_promoter_2 | gGCACGGTTTAATCGCTCCAC | 174 |
| CTCF_promoter_3 | gCAAAGAAGCAGCTCCGCGCA | 175 |
| CTCF_promoter_4 | gGCTCAGACACAAAATGGCGG | 176 |
| CTCF_promoter_5 | gCCCATCGTGACACCTAGAGG | 177 |
| CTCF_promoter_6 | gGCCGCCTCTAGGTGTCACGA | 178 |
| CTCF_promoter_7 | gCGCGAAACCTCCTCCACCCC | 179 |
| CTCF_promoter_8 | gCGCCTCTAGGTGTCACGATG | 180 |
| CTCF_promoter_9 | gGGTGTGGCGCGGAGGTAAGG | 181 |
| CTCF_promoter_10 | gAGGTTTCGCGGGCCGCCTCT | 182 |
| CTCF_promoter_11 | gCGGGGTGGAGGAGGTTTCGC | 183 |
| CTCF_promoter_12 | gGGGTGTGGCGCGGAGGTAAG | 184 |
| CTCF_promoter_13 | gTGGAGCGATTAAACCGTGCG | 185 |
| CTCF_promoter_14 | gCCACAGGCTCAGACACAAAA | 186 |
| CTCF_promoter_15 | gGCGCGGAGCTGCTTCTTTGG | 187 |
| CTCF_promoter_16 | gGCGGTGCAGTGCGCCTGCGT | 188 |
| CTCF_promoter_17 | gGGCCCCGGCAGCGCCGACGC | 189 |
| CTCF_promoter_18 | gCGTGCGCGGAGCTGCTTCTT | 190 |
| CTCF_promoter_19 | gAGCTGCTTCTTTGGCGGCAG | 191 |
| CTCF_promoter_20 | gTGCTTCTTTGGCGGCAGCGG | 192 |
| SafeHarbor_2 | gGTAACCAAGAGTCAGGACTG | 193 |
| SafeHarbor_4 | gGGATCTTATAATCTAGTTAT | 194 |
| SafeHarbor_5 | gGTTAATGCCTTGGTCAAATG | 195 |
| SafeHarbor_7 | gGCTAAAGTTGTCATTGATTT | 196 |
| SafeHarbor_9 | gGGAACGTAGGTAATAAGGTC | 197 |
| SafeHarbor_12 | gGTCAGCATTAAACATGCTTA | 198 |
| SafeHarbor_13 | gGTGAAAGTTCTCATCTTCTT | 199 |
| SafeHarbor_14 | gGCATGAGAAGAGGAGATTGA | 200 |
| SafeHarbor_16 | gGCCCTGTCTGTATCCAGTCC | 201 |
| SafeHarbor_17 | gGGGATCTTTCAGTGTAGGTA | 202 |
| SafeHarbor_18 | gGATTCTGTATAATGGAAATC | 203 |
| SafeHarbor_19 | gGACATGTCCTAATTGTATGG | 204 |
| SafeHarbor_21 | gGCAATATGATCTCATTTGTG | 205 |
| SafeHarbor_22 | gGAGTTTAGAGGTTTGAGATT | 206 |
| SafeHarbor_24 | gGTTATGCCAACACATTTGTA | 207 |

Vectors and virus production-Annealed top and bottom oligos (Table 2) were cloned into an all-in-one KRAB-dCas9-puro vector (pXPR_066, Broad GPP) using the BsmBI restriction enzyme for backbone linearization and T7 ligase for CD81 promoter targeting experiments, the EPB41 enhancer locus experiments, and the CTCF pooled library and follow up. sgCD81i-1 g[9nt] and g[8nt] guides required golden gate assembly (NEB) due to the short length of the oligonucleotides. The CTCF library with varying guide lengths involved the production of 3 separate pooled libraries, one for each of 10nt, 11nt, and 20nt guide lengths. These libraries were then mixed based on molar ratio to produce the final library.

Single plasmids were chemically transformed into One Shot STBL3 chemically competent coli (Invitrogen C737303). Bacterial cultures were shaken at 225 rpm for one hour at 37° C. and then plated on an ampicillin agar dish. After overnight growth at 37° C., single colonies were picked into LB and shaken overnight at 225 rpm and 37° C. Plasmids were isolated the next day using a Plasmid Miniprep Kit (Qiagen) and quantified using a Qubit fluorometer.

Pooled plasmid libraries were electroporated into ElectroMAX™ Stb14™ electrocompetent cells (Invitrogen 11635018) and spread on to bioassay plates. After overnight incubation at 30° C., bacterial colonies were collected and isolated using the Plasmid Plus Midi Kit (Qiagen 12941). 20nt, 11nt, and 10nt sequences of the CTCF pooled library were cloned as individual pools and then combined to reduce drift. Guide sequences can be found in Table 1 above. All guides were cloned with a guanine base at the 5′ end to improve transcription from the U6 promoter (Goa et al. (2017) Transcription, 8(5):275-287; Ma et al. (2014) Molecular Therapy Nucleic Acids, 3(5):e161).

For single plasmids, 1×10⁶ HEK293FT cells were seeded in each 6-well in 2 ml of DMEM+10% FBS 24 hours prior to transfection. A DNA mixture was prepared consisting of 250 μl Opti-MEM, 0.25 μg pCMV_VSVG (Addgene 8454), 1.25 μg psPAX2 (Addgene 12260), 1 μg of the all-in-one CRISPR vector (pXPR_066), and 7.5 ul TransIT-LT1 (Mirus) transfection reagent. After a 20-minute incubation, the solution was added dropwise to the 6-well and incubated for 6-8 hours. Fresh media was added to the cells and collected 36 hours later and either snap frozen or added to cells.

For CTCF pooled library, 8×10⁶ HEK293FT cells were seeded in each of 2 T75 flasks in 12 ml of DMEM+10% FBS 24 hours prior to transfection. Next, pCMV_VSVG (Addgene, 8454, 1.5 μg), psPAX2 (Addgene 12260, 9 μg), the guide containing vector (pXPR_066, 7.5 μg), and 66 μl TransIT-LT1 (Mirus MIR 2306) were combined with 2.1 ml of Opti-MEM to produce TransIT-LT1:DNA complexes. After a 20-minute incubation, the solution was added dropwise to the 6-well and incubated for 6-8 hours, then the media was changed. After 36 hours, the lentivirus was collected, filtered, and either snap frozen or used for cell transduction.

TABLE 2
Top and Bottom Oligos for Preparing KRAB-dCas9-Puro Vectors

| Name | Top Oligo | Bottom Oligo |
|---|---|---|
| Safe Harbor g[20 nt] | caccgGCTAAAGTTGTC | aaacAAATCAATGACAAC |
| | ATTGATTT | TTTAGCc |
| sgCD81i-1 21 nt | caccGGCCTGGCAGGA | aaacCACCGCGCATCCTG |
| | TGCGCGGTG | CCAGGCC |
| sgCD81i-1 20 nt | caccGCCTGGCAGGAT | aaacCACCGCGCATCCTG |
| | GCGCGGTG | CCAGGCC |
| sgCD81i-1 g[18 nt] | caccgCTGGCAGGATGC | aaacCACCGCGCATCCTG |
| | GCGGTG | CCAGc |
| sgCD81i-1 g[17 nt] | caccgTGGCAGGATGCG | aaacCACCGCGCATCCTG |
| | CGGTG | CCAc |
| sgCD81i-1 g[16 nt] | caccgGGCAGGATGCGC | aaacCACCGCGCATCCTG |
| | GGTG | CCc |
| sgCD81i-1 16 nt | caccGGCAGGATGCGC | aaacCACCGCGCATCCTG |
| | GGTG | CC |
| sgCD81i-1 15 nt | caccGCAGGATGCGCG | aaacCACCGCGCATCCTG |
| | GTG | C |
| sgCD81i-1 g[13 nt] | caccgAGGATGCGCGGT | aaacCACCGCGCATCCTc |
| | G | |
| sgCD81i-1 g[12 nt] | caccgGGATGCGCGGTG | aaacCACCGCGCATCCc |
| sgCD81i-1 12 nt | caccGGATGCGCGGTG | aaacCACCGCGCATCC |
| sgCD81i-1 11 nt | caccGATGCGCGGTG | aaacCACCGCGCATC |
| sgCD81i-1 g[9 nt] | gccgtctcgcaccgTGCGCG | gccgtctcgaaacCACCGCGCA |
| | GTGgtttcgagacggc | cggtgcgagacggc |
| sgCD81i-1 g[8 nt] | gccgtctcgcaccgGCGCGG | gccgtctcgaaacCACCGCGCc |
| | TGgtttcgagacggc | ggtgcgagacggc |
| sgCD81i-2 20 nt | caccGCAGCCCTCCACT | aaacCCATGGGAGTGGAG |
| | CCCATGG | GGCTGC |
| sgCD81i-2 17 nt | caccGCCCTCCACTCCC | aaacCCATGGGAGTGGAG |
| | ATGG | GGG |
| sgCD81i-2 g[13 nt] | caccgTCCACTCCCATG | aaacCCATGGGAGTGGAc |
| | G | |
| sgCD81i-2 g[10 nt] | caccgACTCCCATGG | aaacCCATGGGAGTc |
| sgCD81i-3 g[20 nt] | caccgAGAGAGCGAGC | aaacCCGTTGCGCGCTCG |
| | GCGCAACGG | CTCTCTc |
| sgCD81i-3 17 nt | caccGAGCGAGCGCGC | aaacCCGTTGCGCGCTCG |
| | AACGG | CTC |
| sgCD81i-3 g[13 nt] | caccGAGCGCGCAACG | aaacCCGTTGCGCGCTC |
| | G | |
| sgCD81i-3 11 nt | caccGCGCGCAACGG | aaacCCGTTGCGCGC |
| sgCD81-KO-1 g[20 nt] | caccgGTTGACAAAGCC | aaacGCATCTGGGGCTTT |
| | CCAGATGC | GTCAACc |
| sgCD81-KO-1 20 nt | caccGTTGACAAAGCCC | aaacGCATCTGGGGCTTT |
| | CAGATGC | GTCAAC |
| sgCD81-KO-1 g[18 nt] | caccgTGACAAAGCCCC | aaacGCATCTGGGGCTTT |
| | AGATGC | GTCAc |
| sgCD81-KO-1 g[17 nt] | caccgGACAAAGCCCCA | aaacGCATCTGGGGCTTT |
| | GATGC | GTCc |
| sgCD81-KO-1 17 nt | caccGACAAAGCCCCA | aaacGCATCTGGGGCTTT |
| | GATGC | GTC |
| sgCD81-KO-1 g[15 nt] | caccgCAAAGCCCCAGA | aaacGCATCTGGGGCTTT |
| | TGC | Gc |
| sgCD81-KO-2 g[20 nt] | caccgGCGCCCAACACC | aaacACATAGAAGGTGTT |
| | TTCTATGT | GGGCGCc |
| sgCD81-KO-2 20 nt | caccGCGCCCAACACCT | aaacACATAGAAGGTGTT |
| | TCTATGT | GGGCGC |
| sgCD81-KO-2 g[18 nt] | caccgGCCCAACACCTT | aaacACATAGAAGGTGTT |
| | CTATGT | GGGCc |
| sgCD81-KO-2 18 nt | caccGCCCAACACCTTC | aaacACATAGAAGGTGTT |
| | TATGT | GGGC |
| sgCD81-KO-2 g[16 nt] | caccgCCAACACCTTCT | aaacACATAGAAGGTGTT |
| | ATGT | GGc |
| sgCD81-KO-2 g[15 nt] | caccgCAACACCTTCTA | aaacACATAGAAGGTGTT |
| | TGT | Gc |
| EPB41_locus_PU.1 g[20 nt] | caccgAAAAGTTGAGAC | aaacCTTTGTCAGTCTCAA |
| | TGACAAAG | CTTTTc |
| EPB41_locus_PU.1 g[13 nt] | caccgGAGACTGACAAA | aaacCTTTGTCAGTCTCc |
| | G | |
| EPB41_locus_PU.1 11 nt | caccGACTGACAAAG | aaacCTTTGTCAGTC |
| EPB41_locus_SP1 21 nt | caccGGAACAGACAGG | aaacCGCCCCCAACCTGT |
| | TTGGGGGCG | CTGTTCC |
| EPB41_locus_SP1 g[13 nt] | caccgCAGGTTGGGGGC | aaacCGCCCCCAACCTGc |
| | G | |
| EPB41_locus_SP1 11 nt | caccGGTTGGGGGCG | aaacCGCCCCCAACC |
| EPB41_locus_YY1 21 nt | caccGCTTGAGGTGGGT | aaacTCTTTATCACCCACC |
| | GATAAAGA | TCAAGC |
| EPB41_locus_YY1 14 nt | caccGTGGGTGATAAA | aaacTCTTTATCACCCAC |
| | GA | |
| EPB41_locus_YY1 11 nt | caccGGTGATAAAGA | aaacTCTTTATCACC |
| EPB41_locus_NR2 g[20 nt] | caccgATAGAGGGTGTG | aaacGTCACCTTCACACCC |
| | AAGGTGAC | TCTATc |
| EPB41_locus_NR2 14 nt | caccGGTGTGAAGGTG | aaacGTCACCTTCACACC |
| | AC | |
| EPB41_locus_NR2 11 nt | caccGTGAAGGTGAC | aaacGTCACCTTCAC |
| EPB41_validation_enh1 g[20 nt] | caccgGCTTCAGATGGG | aaacACCTCAAGCCCATC |
| | CTTGAGGT | TGAAGCc |
| EPB41_validation_enh2 21 nt | caccgACAGGATAAGG | aaacCCAGGAATTCCTTAT |
| | AATTCCTGG | CCTGTc |
| EPB41_validation_enh3 g[20 nt] | caccgGGAGGTCCAGGC | aaacCTGAGAAGGCCTGG |
| | CTTCTCAG | ACCTCCc |
| EPB41_promoter 21 nt | caccGATTCTGGAATTC | aaacGCGGTCCCGAATTC |
| | GGGACCGC | CAGAATC |
| CTCF_10 nt_sg1 | caccgTGCCCCCTAC | aaacGTAGGGGGCAc |
| CTCF_10 nt_sg2 | caccgTGCCCTCTAC | aaacGTAGAGGGCAc |
| CTCF_10 nt_sg3 | caccgTGCCCCCTTC | aaacGAAGGGGGCAc |
| CTCF_10 nt_sg4 | caccgTGCCATCTAC | aaacGTAGATGGCAc |
| CTCF_10 nt_sg5 | caccgTGCCCCCTTG | aaacCAAGGGGGCAc |
| CTCF_10 nt_sg6 | caccgTGCCCCCTAG | aaacCTAGGGGGCAc |
| CTCF_10 nt_sg7 | caccgTGCCACCTAC | aaacGTAGGTGGCAc |
| CTCF_10 nt_sg8 | caccgTGCCACCTAG | aaacCTAGGTGGCAc |
| CTCF_10 nt_sg9 | caccgTGCCATCTTC | aaacGAAGATGGCAc |
| CTCF_10 nt_sg10 | caccgTGCCCTCTTG | aaacCAAGAGGGCAc |
| CTCF_10 nt_sg11 | caccgTGCCACCTTC | aaacGAAGGTGGCAc |
| CTCF_10 nt_sg12 | caccgTGCCCTCTTC | aaacGAAGAGGGCAc |
| CTCF_10 nt_sg13 | caccgTGCCATCTGG | aaacCCAGATGGCAc |
| CTCF_10 nt_sg14 | caccgTGCCATCTAG | aaacCTAGATGGCAc |
| CTCF_10 nt_sg15 | caccgTGCCATCTTG | aaacCAAGATGGCAc |
| CTCF_10 nt_sg16 | caccgTGCCCTCTAG | aaacCTAGAGGGCAc |
| CTCF_10 nt_sg17 | caccgTGCCACCTTG | aaacCAAGGTGGCAc |
| CTCF_10 nt_sg18 | caccgTGCCCCCTGC | aaacGCAGGGGGCAc |
| CTCF_10 nt_sg19 | caccgTGCCACCTGG | aaacCCAGGTGGCAc |
| CTCF_10 nt_sg20 | caccgTGCCATCTGC | aaacGCAGATGGCAc |
| CTCF_10 nt_sg21 | caccgTGCCCCCTGG | aaacCCAGGGGGCAc |
| CTCF_10 nt_sg22 | caccgTGCCCTCTGG | aaacCCAGAGGGCAc |
| CTCF_10 nt_sg23 | caccgTGCCACCTGC | aaacGCAGGTGGCAc |
| CTCF_10 nt_sg24 | caccgTGCCCTCTGC | aaacGCAGAGGGCAc |
| CTCF_gaag[sg4] | caccgaagTGCCATCTAC | aaacGTAGATGGCActtc |
| CTCF_1_10 nt + A | caccgATGCCCCCTAC | aaacGTAGGGGGCATc |
| CTCF_1_10 nt + T | caccgTTGCCCCCTAC | aaacGTAGGGGGCAAc |
| CTCF_1_10 nt + G | caccgGTGCCCCCTAC | aaacGTAGGGGGCACc |
| CTCF_1_10 nt + C | caccgCTGCCCCCTAC | aaacGTAGGGGGCAGc |
| CTCF_2_10 nt + A | caccgATGCCCTCTAC | aaacGTAGAGGGCATc |
| CTCF_2_10 nt + T | caccgTTGCCCTCTAC | aaacGTAGAGGGCAAc |
| CTCF_2_10 nt + G | caccgGTGCCCTCTAC | aaacGTAGAGGGCACc |
| CTCF_2_10 nt + C | caccgCTGCCCTCTAC | aaacGTAGAGGGCAGc |
| CTCF_3_10 nt + A | caccgATGCCCCCTTC | aaacGAAGGGGGCATc |
| CTCF_3_10 nt + T | caccgTTGCCCCCTTC | aaacGAAGGGGGCAAc |
| CTCF_3_10 nt + G | caccgGTGCCCCCTTC | aaacGAAGGGGGCACc |
| CTCF_3_10 nt + C | caccgCTGCCCCCTTC | aaacGAAGGGGGCAGc |
| CTCF_4_10 nt + A | caccgATGCCATCTAC | aaacGTAGATGGCATc |
| CTCF_4_10 nt + T | caccgTTGCCATCTAC | aaacGTAGATGGCAAc |
| CTCF_4_10 nt + G | caccgGTGCCATCTAC | aaacGTAGATGGCACc |
| CTCF_4_10 nt + C | caccgCTGCCATCTAC | aaacGTAGATGGCAGc |
| CTCF_5_10 nt + A | caccgATGCCCCCTTG | aaacCAAGGGGGCATc |
| CTCF_5_10 nt + T | caccgTTGCCCCCTTG | aaacCAAGGGGGCAAc |
| CTCF_5_10 nt + G | caccgGTGCCCCCTTG | aaacCAAGGGGGCACc |
| CTCF_5_10 nt + C | caccgCTGCCCCCTTG | aaacCAAGGGGGCAGc |
| CTCF_6_10 nt + A | caccgATGCCCCCTAG | aaacCTAGGGGGCATc |
| CTCF_6_10 nt + T | caccgTTGCCCCCTAG | aaacCTAGGGGGCAAc |
| CTCF_6_10 nt + G | caccgGTGCCCCCTAG | aaacCTAGGGGGCACc |
| CTCF_6_10 nt + C | caccgCTGCCCCCTAG | aaacCTAGGGGGCAGc |
| CTCF_7_10 nt + A | caccgATGCCACCTAC | aaacGTAGGTGGCATc |
| CTCF_7_10 nt + T | caccgTTGCCACCTAC | aaacGTAGGTGGCAAc |
| CTCF_7_10 nt + G | caccgGTGCCACCTAC | aaacGTAGGTGGCACc |
| CTCF_7_10 nt + C | caccgCTGCCACCTAC | aaacGTAGGTGGCAGc |
| CTCF_8_10 nt + A | caccgATGCCACCTAG | aaacCTAGGTGGCATc |
| CTCF_8_10 nt + T | caccgTTGCCACCTAG | aaacCTAGGTGGCAAc |
| CTCF_8_10 nt + G | caccgGTGCCACCTAG | aaacCTAGGTGGCACc |
| CTCF_8_10 nt + C | caccgCTGCCACCTAG | aaacCTAGGTGGCAGc |
| CTCF_9_10 nt + A | caccgATGCCATCTTC | aaacGAAGATGGCATc |
| CTCF_9_10 nt + T | caccgTTGCCATCTTC | aaacGAAGATGGCAAc |
| CTCF_9_10 nt + G | caccgGTGCCATCTTC | aaacGAAGATGGCACc |
| CTCF_9_10 nt + C | caccgCTGCCATCTTC | aaacGAAGATGGCAGc |
| CTCF_10_10 nt + A | caccgATGCCCTCTTG | aaacCAAGAGGGCATc |
| CTCF_10_10 nt + T | caccgTTGCCCTCTTG | aaacCAAGAGGGCAAc |
| CTCF_10_10 nt + G | caccgGTGCCCTCTTG | aaacCAAGAGGGCACc |
| CTCF_10_10 nt + C | caccgCTGCCCTCTTG | aaacCAAGAGGGCAGc |
| CTCF_11_10 nt + A | caccgATGCCACCTTC | aaacGAAGGTGGCATc |
| CTCF_11_10 nt + T | caccgTTGCCACCTTC | aaacGAAGGTGGCAAC |
| CTCF_11_10 nt + G | caccgGTGCCACCTTC | aaacGAAGGTGGCACc |
| CTCF_11_10 nt + C | caccgCTGCCACCTTC | aaacGAAGGTGGCAGc |
| CTCF_12_10 nt + A | caccgATGCCCTCTTC | aaacGAAGAGGGCATc |
| CTCF_12_10 nt + T | caccgTTGCCCTCTTC | aaacGAAGAGGGCAAc |
| CTCF_12_10 nt + G | caccgGTGCCCTCTTC | aaacGAAGAGGGCACc |
| CTCF_12_10 nt + C | caccgCTGCCCTCTTC | aaacGAAGAGGGCAGc |
| CTCF_13_10 nt + A | caccgATGCCATCTGG | aaacCCAGATGGCATc |
| CTCF_13_10 nt + T | caccgTTGCCATCTGG | aaacCCAGATGGCAAc |
| CTCF_13_10 nt + G | caccgGTGCCATCTGG | aaacCCAGATGGCACc |
| CTCF_13_10 nt + C | caccgCTGCCATCTGG | aaacCCAGATGGCAGc |
| CTCF_14_10 nt + A | caccgATGCCATCTAG | aaacCTAGATGGCATc |
| CTCF_14_10 nt + T | caccgTTGCCATCTAG | aaacCTAGATGGCAAc |
| CTCF_14_10 nt + G | caccgGTGCCATCTAG | aaacCTAGATGGCACc |
| CTCF_14_10 nt + C | caccgCTGCCATCTAG | aaacCTAGATGGCAGc |
| CTCF_15_10 nt + A | caccgATGCCATCTTG | aaacCAAGATGGCATc |
| CTCF_15_10 nt + T | caccgTTGCCATCTTG | aaacCAAGATGGCAAc |
| CTCF_15_10 nt + G | caccgGTGCCATCTTG | aaacCAAGATGGCACc |
| CTCF_15_10 nt + C | caccgCTGCCATCTTG | aaacCAAGATGGCAGc |
| CTCF_16_10 nt + A | caccgATGCCCTCTAG | aaacCTAGAGGGCATc |
| CTCF_16_10 nt + T | caccgTTGCCCTCTAG | aaacCTAGAGGGCAAc |
| CTCF_16_10 nt + G | caccgGTGCCCTCTAG | aaacCTAGAGGGCACc |
| CTCF_16_10 nt + C | caccgCTGCCCTCTAG | aaacCTAGAGGGCAGc |
| CTCF_17_10 nt + A | caccgATGCCACCTTG | aaacCAAGGTGGCATc |
| CTCF_17_10 nt + T | caccgTTGCCACCTTG | aaacCAAGGTGGCAAc |
| CTCF_17_10 nt + G | caccgGTGCCACCTTG | aaacCAAGGTGGCACc |
| CTCF_17_10 nt + C | caccgCTGCCACCTTG | aaacCAAGGTGGCAGc |
| CTCF_18_10 nt + A | caccgATGCCCCCTGC | aaacGCAGGGGGCATc |
| CTCF_18_10 nt + T | caccgTTGCCCCCTGC | aaacGCAGGGGGCAAc |
| CTCF_18_10 nt + G | caccgGTGCCCCCTGC | aaacGCAGGGGGCACc |
| CTCF_18_10 nt + C | caccgCTGCCCCCTGC | aaacGCAGGGGGCAGc |
| CTCF_19_10 nt + A | caccgATGCCACCTGG | aaacCCAGGTGGCATc |
| CTCF_19_10 nt + T | caccgTTGCCACCTGG | aaacCCAGGTGGCAAc |
| CTCF_19_10 nt + G | caccgGTGCCACCTGG | aaacCCAGGTGGCACc |
| CTCF_19_10 nt + C | caccgCTGCCACCTGG | aaacCCAGGTGGCAGc |
| CTCF_20_10 nt + A | caccgATGCCATCTGC | aaacGCAGATGGCATc |
| CTCF_20_10 nt + T | caccgTTGCCATCTGC | aaacGCAGATGGCAAc |
| CTCF_20_10 nt + G | caccgGTGCCATCTGC | aaacGCAGATGGCACc |
| CTCF_20_10 nt + C | caccgCTGCCATCTGC | aaacGCAGATGGCAGc |
| CTCF_21_10 nt + A | caccgATGCCCCCTGG | aaacCCAGGGGGCATc |
| CTCF_21_10 nt + T | caccgTTGCCCCCTGG | aaacCCAGGGGGCAAc |
| CTCF_21_10 nt + G | caccgGTGCCCCCTGG | aaacCCAGGGGGCACc |
| CTCF_21_10 nt + C | caccgCTGCCCCCTGG | aaacCCAGGGGGCAGc |
| CTCF_22_10 nt + A | caccgATGCCCTCTGG | aaacCCAGAGGGCATc |
| CTCF_22_10 nt + T | caccgTTGCCCTCTGG | aaacCCAGAGGGCAAc |
| CTCF_22_10 nt + G | caccgGTGCCCTCTGG | aaacCCAGAGGGCACc |
| CTCF_22_10 nt + C | caccgCTGCCCTCTGG | aaacCCAGAGGGCAGc |
| CTCF_23_10 nt + A | caccgATGCCACCTGC | aaacGCAGGTGGCATc |
| CTCF_23_10 nt + T | caccgTTGCCACCTGC | aaacGCAGGTGGCAAc |
| CTCF_23_10 nt + G | caccgGTGCCACCTGC | aaacGCAGGTGGCACc |
| CTCF_23_10 nt + C | caccgCTGCCACCTGC | aaacGCAGGTGGCAGc |
| CTCF_24_10 nt + A | caccgATGCCCTCTGC | aaacGCAGAGGGCATc |
| CTCF_24_10 nt + T | caccgTTGCCCTCTGC | aaacGCAGAGGGCAAc |
| CTCF_24_10 nt + G | caccgGTGCCCTCTGC | aaacGCAGAGGGCACc |
| CTCF_24_10 nt + C | caccgCTGCCCTCTGC | aaacGCAGAGGGCAGc |
| CTCF_promoter_1 | caccgCAGGCTCAGACA | aaacCCATTTTGTGTCTGA |
| | CAAAATGG | GCCTGc |
| CTCF_promoter_2 | caccgGCACGGTTTAAT | aaacGTGGAGCGATTAAA |
| | CGCTCCAC | CCGTGCc |
| CTCF_promoter_3 | caccgCAAAGAAGCAG | aaacTGCGCGGAGCTGCT |
| | CTCCGCGCA | TCTTTGc |
| CTCF_promoter_4 | caccgGCTCAGACACAA | aaacCCGCCATTTTGTGTC |
| | AATGGCGG | TGAGCc |
| CTCF_promoter_5 | caccgCCCATCGTGACA | aaacCCTCTAGGTGTCAC |
| | CCTAGAGG | GATGGGc |
| CTCF_promoter_6 | caccgGCCGCCTCTAGG | aaacTCGTGACACCTAGA |
| | TGTCACGA | GGCGGCc |
| CTCF_promoter_7 | caccgCGCGAAACCTCC | aaacGGGGTGGAGGAGGT |
| | TCCACCCC | TTCGCGc |
| CTCF_promoter_8 | caccgCGCCTCTAGGTG | aaacCATCGTGACACCTA |
| | TCACGATG | GAGGCGc |
| CTCF_promoter_9 | caccgGGTGTGGCGCGG | aaacCCTTACCTCCGCGCC |
| | AGGTAAGG | ACACCc |
| CTCF_promoter_10 | caccgAGGTTTCGCGGG | aaacAGAGGCGGCCCGCG |
| | CCGCCTCT | AAACCTc |
| CTCF_promoter_11 | caccgCGGGGTGGAGG | aaacGCGAAACCTCCTCC |
| | AGGTTTCGC | ACCCCGc |
| CTCF_promoter_12 | caccgGGGTGTGGCGCG | aaacCTTACCTCCGCGCCA |
| | GAGGTAAG | CACCCc |
| CTCF_promoter_13 | caccgTGGAGCGATTAA | aaacCGCACGGTTTAATC |
| | ACCGTGCG | GCTCCAc |
| CTCF_promoter_14 | caccgCCACAGGCTCAG | aaacTTTTGTGTCTGAGCC |
| | ACACAAAA | TGTGGc |
| CTCF_promoter_15 | caccgGCGCGGAGCTGC | aaacCCAAAGAAGCAGCT |
| | TTCTTTGG | CCGCGCc |
| CTCF_promoter_16 | caccgGCGGTGCAGTGC | aaacACGCAGGCGCACTG |
| | GCCTGCGT | CACCGCc |
| CTCF_promoter_17 | caccgGGCCCCGGCAGC | aaacGCGTCGGCGCTGCC |
| | GCCGACGC | GGGGCCc |
| CTCF_promoter_18 | caccgCGTGCGCGGAGC | aaacAAGAAGCAGCTCCG |
| | TGCTTCTT | CGCACGc |
| CTCF_promoter_19 | caccgAGCTGCTTCTTT | aaacCTGCCGCCAAAGAA |
| | GGCGGCAG | GCAGCTc |
| CTCF_promoter_20 | caccgTGCTTCTTTGGC | aaacCCGCTGCCGCCAAA |
| | GGCAGCGG | GAAGCAc |
| SafeHarbor_2 | caccgGTAACCAAGAGT | aaacCAGTCCTGACTCTTG |
| | CAGGACTG | GTTACc |
| SafeHarbor_4 | caccgGGATCTTATAAT | aaacATAACTAGATTATA |
| | CTAGTTAT | AGATCCc |
| SafeHarbor_5 | caccgGTTAATGCCTTG | aaacCATTTGACCAAGGC |
| | GTCAAATG | ATTAACc |
| SafeHarbor_7 | caccgGCTAAAGTTGTC | aaacAAATCAATGACAAC |
| | ATTGATTT | TTTAGCc |
| SafeHarbor_9 | caccgGGAACGTAGGTA | aaacGACCTTATTACCTAC |
| | ATAAGGTC | GTTCCc |
| SafeHarbor_12 | caccgGTCAGCATTAAA | aaacTAAGCATGTTTAAT |
| | CATGCTTA | GCTGACc |
| SafeHarbor_13 | caccgGTGAAAGTTCTC | aaacAAGAAGATGAGAAC |
| | ATCTTCTT | TTTCACc |
| SafeHarbor_14 | caccgGCATGAGAAGA | aaacTCAATCTCCTCTTCT |
| | GGAGATTGA | CATGCc |
| SafeHarbor_16 | caccgGCCCTGTCTGTA | aaacGGACTGGATACAGA |
| | TCCAGTCC | CAGGGCc |
| SafeHarbor_17 | caccgGGGATCTTTCAG | aaacTACCTACACTGAAA |
| | TGTAGGTA | GATCCCc |
| SafeHarbor_18 | caccgGATTCTGTATAA | aaacGATTTCCATTATACA |
| | TGGAAATC | GAATCc |
| SafeHarbor_19 | caccgGACATGTCCTAA | aaacCCATACAATTAGGA |
| | TTGTATGG | CATGTCc |
| SafeHarbor_21 | caccgGCAATATGATCT | aaacCACAAATGAGATCA |
| | CATTTGTG | TATTGCc |
| SafeHarbor_22 | caccgGAGTTTAGAGGT | aaacAATCTCAAACCTCT |
| | TTGAGATT | AAACTCc |
| SafeHarbor_24 | caccgGTTATGCCAACA | aaacTACAAATGTGTTGG |
| | CATTTGTA | CATAACc |

Viral transduction-CTCF pooled library frozen viral supernatant (300 μl) was thawed and added to 700 μl target cells in 12-wells with a final volume of 10 μg/mL polybrene, resulting in a 30-50% transduction efficiency, corresponding to an MOI of ~0.35-0.70. Cells with viral supernatant were centrifuged at 2000×g for 20 minutes at 22° C. and incubated overnight. After 18-24 hours, cells were fed fresh media and maintained at 2×10⁵ cells/mL for suspension cells (Jurkat, K562, and MV4-11) and 1-2×10⁵ cells/cm² for adherent cells (A375 and HEK293). Cells were passaged into media supplemented with 1 μg/mL puromycin 3 days after transduction. Seven days after transduction, cells were passaged into 0.5 μg/mL puromycin (½ dose) and cultured continuously for the duration of the screen. At day 7, 14, and 21, pellets of 1×10⁶ cells were snap frozen on dry ice and stored at −80° C. in preparation for gDNA isolation.

Single transductions were performed identically to the pooled production, with the exception that viral supernatant varied based on viral titer.

Genomic DNA preparation and sequencing—Genomic DNA was isolated using DNeasy Blood and Tissue Kit (Qiagen 69504). PCR, sequence adaptor barcoding, cleanup, sequencing, and data deconvolution were carried out as previously described (Najm et al. (2017) Nature Biotechnology, 36(2), 179-189). PCR primers were Argon and Beaker (Broad Institute GPP). At the PCR stage, CTCF pooled library plasmid DNA (pDNA) was diluted to 10 ng for amplification. All PCR reactions were carried out for 28 cycles.

Libraries were prepared using TruSeq amplicon construction and single end sequenced on a MiSeq50. Fastq files were deconvolved using PoolQ (Broad Institute GPP). Apron (Broad Institute GPP) was used to analyze the distribution of each guide relative to the plasmid DNA, enabling enrichment/depletion measurements.

ChIP-seq sample preparation—Frozen crosslinked cell pellets (1×10⁷ cells) were suspended in cell lysis buffer (20 mM Tris pH 8.0, 85 mM KCl, 0.5% NP40) with protease inhibitors (complete EDTA-free Protease Inhibitor Tablets, Sigma Aldrich 4693132001), incubated on ice for 10 min, then centrifuged at 1000×g for 5 minutes. Cell pellets were resuspended for a second time in cell lysis buffer with protease inhibitors, incubated on ice for 5 minutes and centrifuged for 5 minutes at 1000×g. The pellets were resuspended in nuclear lysis buffer (10 mM Tris-HCl pH7.5, 1% NP40, 0.5% sodium deoxycholate, 0.1% SDS) with protease inhibitors for 10 min and subsequently sheared in a sonifier (Branson).

The chromatin was quantified after sonication to determine the cell number in each sample. H3K9me3 ChIP-seq samples were prepared with 1.5×10⁶ cells and 0.4 μg H3K9me3 antibody (Abcam ab176916). CTCF ChIP-seq samples were prepared with 3×10⁶ cells and 1 μg CTCF antibody (Diagenode C15410210). ChIP-seq Dilution Buffer (16.7 mM Tris-HCl pH 8.1, 167 mM NaCl, 0.01% SDS, 1.1% Triton X-100, 1.2 mM EDTA) with protease inhibitors was added to bring the ChIP volume to 0.5 mL. ChIP-seq samples were rotated overnight at 4° C. The following day, Protein A Dynabeads (Invitrogen) were added for 1 hour to enrich fragments of interest. The ChIP-seq samples were removed from rotation and placed on a magnet to isolate the beads. The beads were washed with a series of buffers, low salt RIPA buffer, high salt RIPA buffer, LiCl buffer (250 mM LiCl, 0.5% NP40, 0.5% sodium deoxycholate, 1 mM EDTA, 10 mM Tris-HCl pH 8.1) and finally Low TE. The Protein A beads were then suspended in 50 μl elution buffer (10 mM Tris-Cl pH 8.0, 5 mM EDTA, 300 mM NaCl, 0.1% SDS and 5 mM DTT directly before use) and 8 μl of reverse crosslinking mix (250 mM Tris-HCl pH 6.5, 1.25 M NaCl, 62.5 mM EDTA, 5 mg/ml Proteinase K, and 62.5 μg/ml RNAse A). The suspended beads were incubated at 65° C. for a minimum of 3 hours. After incubation, the supernatants were transferred to a clean tube. The DNA was SPRI purified, eluted, and quantified by Qubit. Libraries with 6 μg of input were prepared using the KAPA Hyper Prep Kit.

Quantitative real time PCR-Real time PCR was performed as described previously Najm et al. (2023) Nature Communications, 14(1):448. In brief, RNA extraction was performed with the RNeasy Plus Micro Kit (Qiagen) and cDNA was synthesized using the Superscript III First-Strand Synthesis System for RT-PCR (Invitrogen). Probes for EPB41 and actin beta transcripts (EPB41_1_For AACTTCCCAGTTACCGAGCA, EPB41_1_Rev CTTGAGTCCGGCCACTGTAT, EPB41_2 For CTGCTCTAGTGGCCTTCTGG, EPB41 2 Rev CTGCTCGGTAACTGGGAAGT, actin-b_For CATCGAGCACGGCATCGTCA, and actin-b Rev TAGCACAGCCTGGATAGCAAC) were paired with Power SYBR Green PCR Master Mix (Applied Biosystems) for quantification. Samples were analyzed on a BioRad CFX Opus 384 Real-Time PCR System. All samples were normalized to the average Ct across all replicates of actin beta safe harbor.

RNA sequencing sample preparation and analysis-Jurkat and A375 cells were transduced with all-in-one KRAB-dCas9-puro (pXPR_066) vector carrying safe harbor, CD81 CRISPRi (20nt or g[9nt]), CTCF-sg4 (10nt), or CTCF-sg8 (10nt) guides (Table 1) were pelleted and stored in −80° C. RNA was isolated with RNeasy Plus Micro kit (Qiagen) according to the manufacturer's protocol and ensuring RIN values greater than 7. Libraries were prepared first with Poly-A enrichment using magnetic oligo (dT)-beads (Invitrogen), then ligated to RNA adaptors for sequencing. Paired end sequencing (2×150 bp) was carried out on an Illumina Nextseq or Novaseq (Illumina).

### Data Analysis

Pooled screening-Log fold change calculations were calculated with the starting plasmid DNA pool as reference. Initial quality control of pooled screening data included running pairwise comparisons on replicates to assess replicate consistency. Based on these tests, one replicate of the screen in K562 cells was excluded, as multiple comparisons test of these replicates identified a significant difference between replicate LFC values (Repeated measures one-way ANOVA, p<0.0001) and a post-hoc multiple comparisons test identified significant differences between Rep A and Rep B as well as between Rep B and Rep C (Tukey's, p<0.0001 for both tests) while there was no significant difference between Rep A and Rep C (Tukey's, p=0.794). Based on these findings, Rep B was excluded from further analysis while the 2 other replicates from this screen were retained. All other cell line replicates were not significantly different.

Z-scores for pooled screen analysis were calculated using the following equation:

$$\frac{gRN {log}_{2} fold change - mean ( safe harbor {log}_{2} fold change )}{standard deviation of safe harbor {log}_{2} fold change}$$

RNA-seq data processing—RNA-seq data for A375 sgCD81, Jurkat sg4 and sg8 were processed using the Kallisto v0.46.1 alignment and quantification tool (kallisto quant-i {transcriptome_index} -o output -b 50 ~{read1} ~{read2} -t 4 -g {gtf}). Transcriptome index and gtf for human was taken from github.com/pachterlab/kallisto-transcriptome-indices/releases. The data, composed of paired-end reads in fastq format and included multiple replicates per condition. Output files of interest to this analysis were the .h5 count matrices and alignment logs.

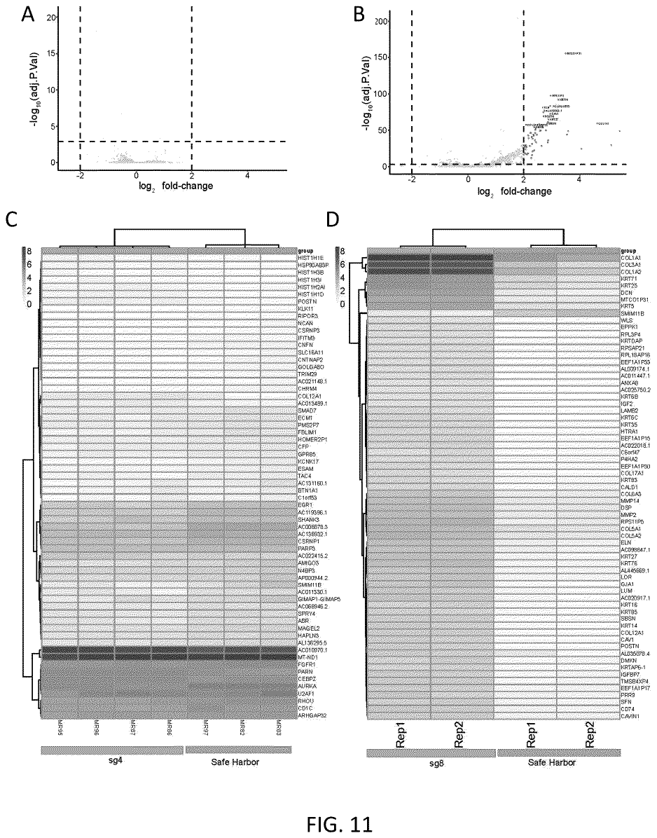

RNA-seq quantification and analysis—The .h5 count matrices were imported into R using tximport::tximport. Raw counts were aggregated to the gene level. Genes with zero counts across all samples were subsequently removed from the matrix for differential expression analysis. Differential expression followed the workflow outlined in the vignette provided within the dream (Hoffman & Roussos, 2021) statistical package. To visualize the impact of the guide in comparison to the Safe Harbor, volcano plots were generated (FIGS. 2D and 11 A-B). The significance criteria was set to have an absolute log fold-change (abs(log FC))>2 and an adjusted p-value (adj. pval)<1e-3. The top differentially expressed genes were visualized through heatmaps using pheatmap::pheatmap, with hierarchical clustering revealing that replicates clustered together.

Comparison of CTCF guide target sites and CTCF binding events-Perfect match sites were identified using Cas-OFFinder v2.4 (Bae et al. (2014) Bioinformatics, 30(1):1473-1475) with hg38 2 bit as reference. Alt chromosomal matches were excluded from the analysis. “N” bases were added to guide sequences such that they met the minimum threshold of 15 nt.

Putative CTCF binding site determination—CTCF sites were selected using the JASPAR MA0139 matrix profile and filtered down to the 880k sites using the R library and steps previously detailed Dozmorov et al. (2022) Bioinformatics Advances, 2(1):vbac097). These were exported to a .bed file and a .saf file.

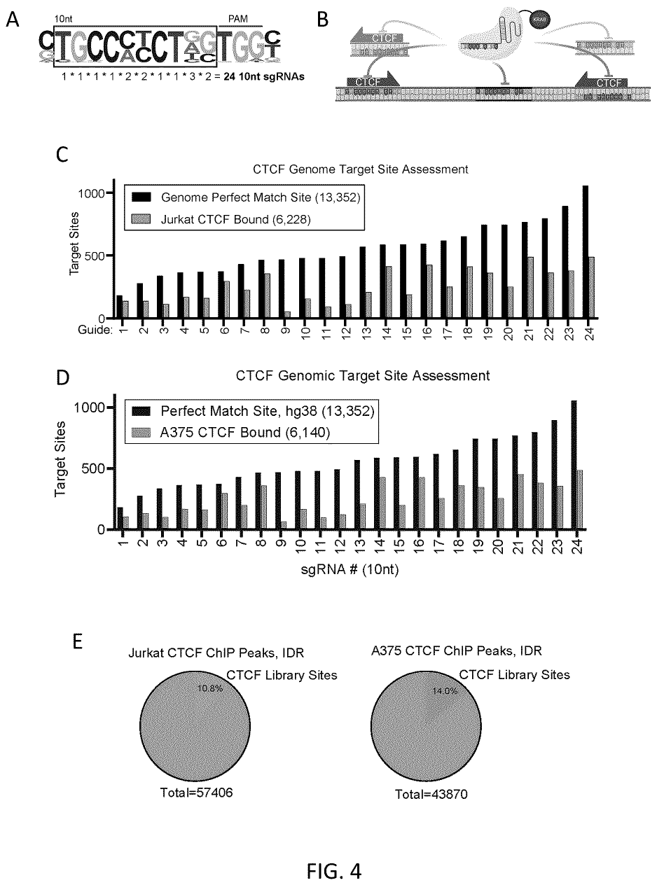

ChIP-seq data processing—For FIG. 4C-E, Jurkat and A375 CTCF ChIP-seq datasets were processed using the ENCODE ChIP-seq pipeline v2.1.5. Both replicates were processed using the default “tf” options for the pipeline with the MACS2 peak-caller. To obtain a final peak-set of CTCF binding events, the IDR-optimal output peak calls at an IDR threshold of <0.05 was utilized for each cell line and merged overlapping peaks. The CTCF peaks were then extended symmetrically by +/−50 bp, corresponding to a stringent perturbation radius, and used bedtools to obtain overlapping sites between each CTCF guide target site and CTCF binding peaks.

For all other figures, ChIP-seq data for CTCF and H3K9me3 were processed using the ENCODE ChIP-seq pipeline v2.2.0 with default parameters. The pipeline_type parameter for CTCF and H3K9me3 was set to “tf” and “histone”, respectively. The data, composed of single-end reads in fastq format, included two replicates per condition. Output files of interest to this analysis were the bam files and QC html reports.

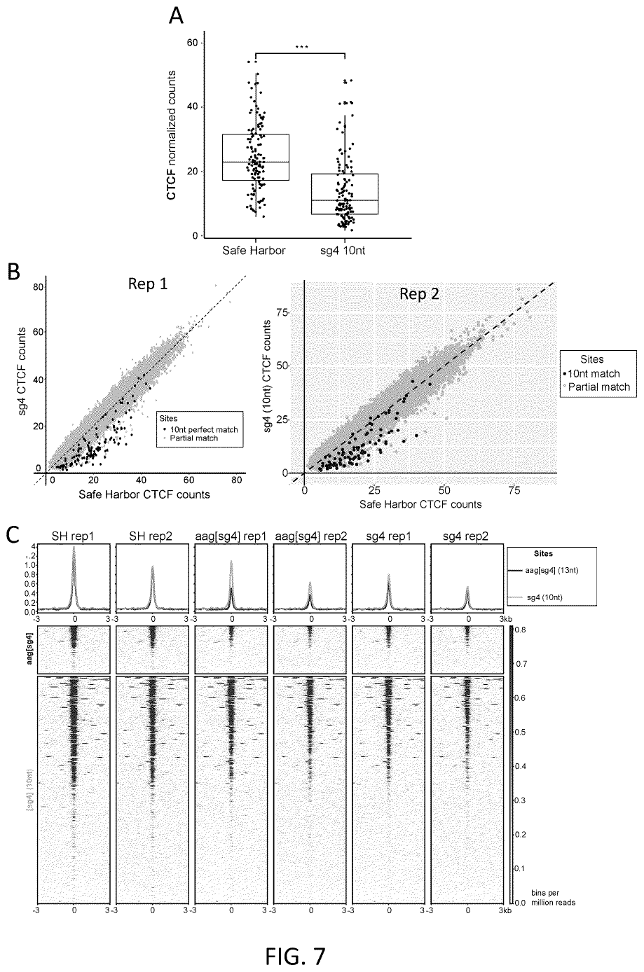

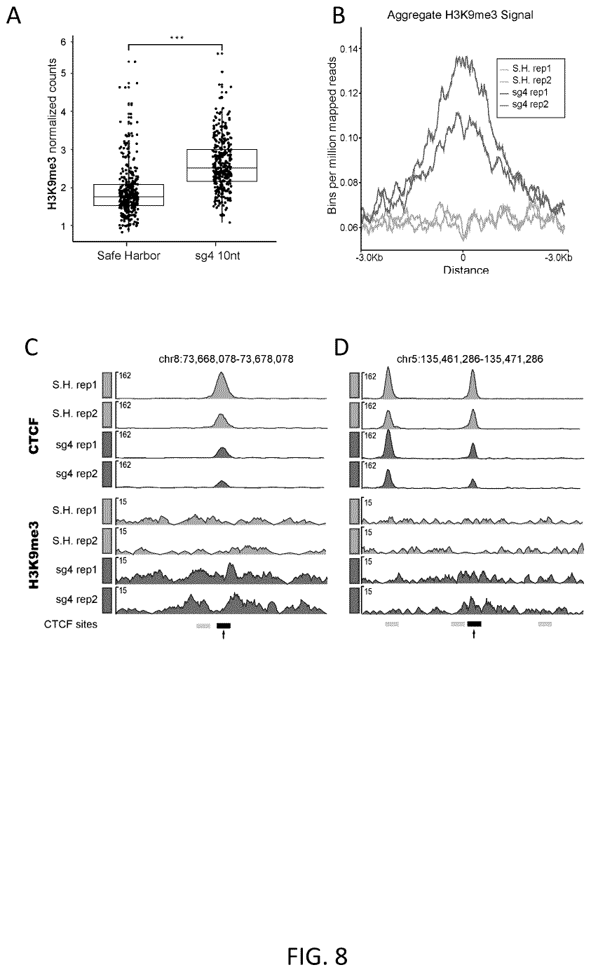

ChIP-seq quantification and analysis—CTCF bigwig files were created using bamCoverage (bamCoverage-b $1 -o “$2.bw” -bs 50 -p 4—effectiveGenomeSize 2913022398—normalizeUsing bpm). Deeptools was used to calculate normalized signal using counts within a +/−3Kb window centered at perfect match sites. To observe the effect of the guide in the H3K9me3 landscape, the windows overlapping perfect match sites were extracted and the aggregate histone signal in Safe Harbor and sg4 samples was plotted (FIG. 8B). CTCF profile heatmaps were also generated (FIG. 7C).

The bigwigs were used with the karyoploteR R package to generate genome tracks (FIGS. 8C-D).

For visualization and differential binding analysis, a CTCF count matrix was created using featureCounts (featureCounts (files, allowMultiOverlap=T, largestOverlap=T, annot.ext=“jaspar_motifs.saf”, readExtension3=200, ignoreDup=T), where jaspar_motifs.saf contains the putative CTCF binding sites with window size 500 bp in a.saf format. The same was done for a H3K9me3 count matrix, except the .saf file contains genome-wide non-overlapping windows of size 5Kb. Raw counts were stored in a .tsv file.

To observe the effect of the guide in CTCF binding at perfect match sites, the count matrix was first loaded in R (rows: ~880k putative binding sites from JASPAR, columns: samples) and used edgeR::cpm to normalize the data, the bins were extracted that overlapped a perfect match site and filtered out if they had <5 CPM in the Safe Harbor samples. CTCF binding was averaged between sample replicates. A significant difference in mean CTCF binding between Safe Harbor and sg4 samples was observed, using stats::t.test (FIGS. 7A, 9, and 10A-B). Similar procedure was used for H3K9me3 (FIG. 8A).

To observe the genome-wide effect of the guide in CTCF binding, the above normalized CTCF count matrix was taken and filtered out bins if they had <5 CPM in the Safe Harbor samples. The counts were plotted in the remaining bins and dots if they overlap a sg4 or aag[sg4] perfect match site (FIGS. 7B, 9, and 10C).

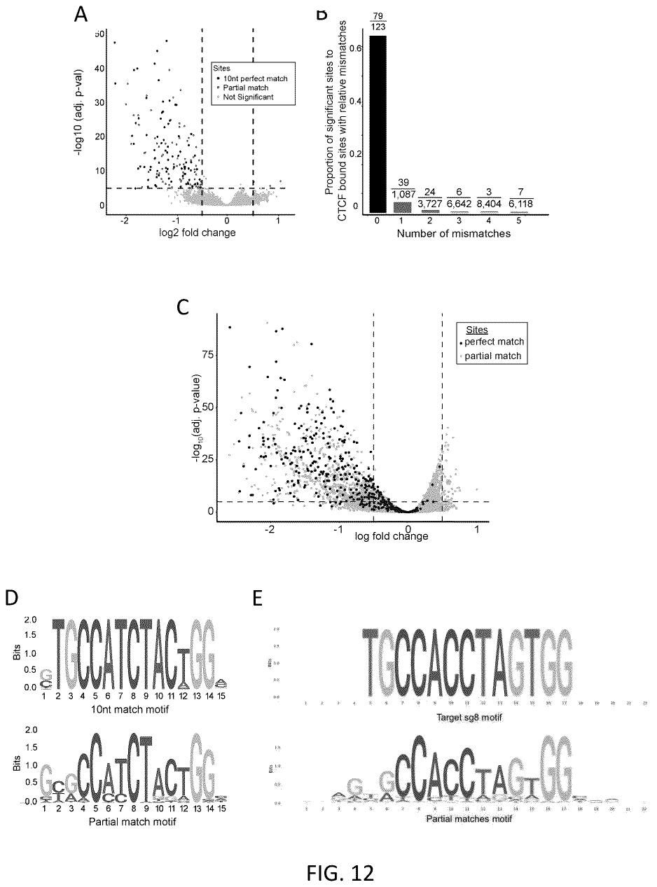

For the differential analysis, the dream statistical package was utilized in R, as outlined by Hoffman and Roussos, 2021. Significance criteria was established with a requirement for absolute log fold change (abs(log FC))>0.5 and an adjusted p-value (adj. p. val)<1e-5. (FIGS. 12A and C). While many significant sites overlapped perfect match sites, some sites were also observed that showed significant CTCF loss where the sequence did not perfectly match the guide target sequence. The sequence was extracted at these sites from JASPAR MA0139, and used Biostrings::consensusMatrix and ggseqlogo::ggseqlogo to look at the logogram of the sequences (FIGS. 12D-E). The sequences matched closely with the guide target sequence. Further analysis showed most of these sequences had only 1 or 2 mismatches from the target sequence (FIG. 12B).

R (version 4.1.2), Python (version 3.7), and Graphpad Prism (version 10) were used for visualization.

### Example 1—Truncated Guides Direct CRISPRi to a Sequence Match Site

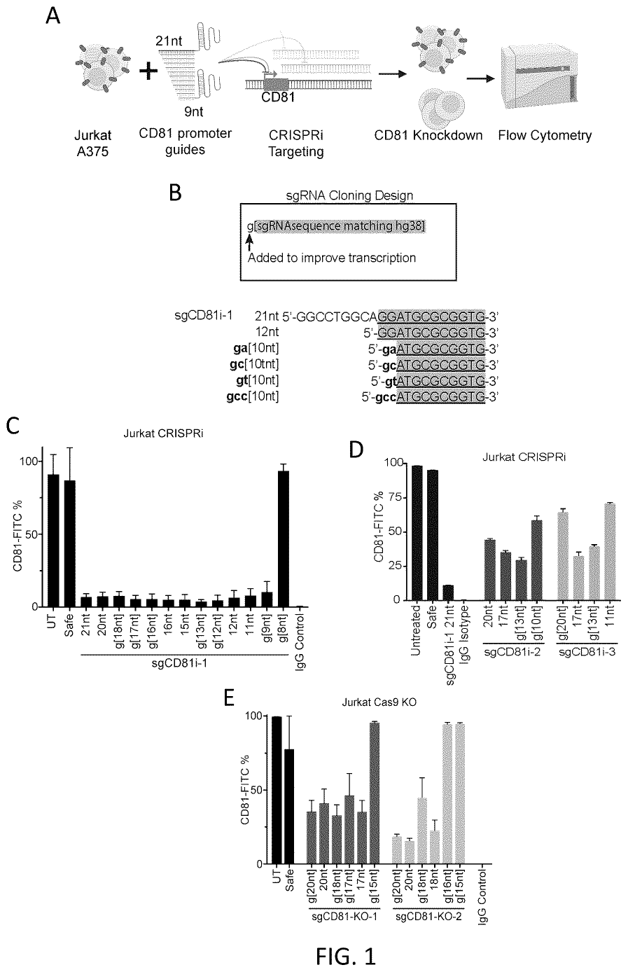

Guide length requirements for CRISPRi-mediated repression were first characterized. CD81, a stably expressed, non-essential cell-surface protein, served as a reporter of on-target efficiency by flow cytometry (FIG. 1A). High performing 20nt S. pyogenes spacer (sgCD81i-1) (Sanson et al. (2018) Nature Communications, 9(1):5416) was selected, directed to the CD81 transcriptional start site (TSS) and tested sequential truncations. By convention, guides were cloned with a guanine in the 5′ position to improve Pol III transcription levels (Ma et al. (2014) Molecular Therapy Nucleic Acids, 3(5):e161), sometimes resulting in the guanine complementing the target sequence (see, Methods and FIG. 1B). Therefore, here brackets were used to denote the length of guide sequence that complements a single target site. For example, sgCD81i-1 g[12nt] consists of a 5′ mismatched guanine and 12 complementary bases to the CD81 TSS. Sequential 5′ truncations of sgCD81i-1 resulted in repression with each guide down to 9nt target match in Jurkat (T lymphocyte) cells (FIG. 1C), with sgCD81i-1 g[9nt] showing activity while sgCD81i-1 g[8nt] guide showed a complete loss of on-target activity. Next additional CD81 TSS 20nt guides with less effective on-target efficiency were tested (sgCD81i-2 and sgCD81i-3). These truncated guides resulted in similar and sometimes better CD81 repression relative to the respective 20nt guide (FIG. 1D). It was expected that CRISPR knockout would be ineffective with sizeable truncations based on prior studies (Cencic et al. (2014) PLOS One, 9(10):e109213; Fu et al. (2014), Nature Biotechnology, 32(3):279-284; Zhang et al. (2016) Scientific Reports, 6(1)). Accordingly, two guides were designed that target exon 1 of CD81 (sgCD81-KO-1 and -2). CD81 knockout was effective at lengths down to a 17nt target match, consistent with prior findings (FIG. 1E). Indeed, guide length requirements for Cas9 cleavage and CRISPRi diverge at <17nt guide lengths, highlighting opportunities for CRISPRi targeting with truncated guides that are not possible with Cas9 cleavage.

Next, the specificity of truncated guide repression was examined. Unpaired bases at the 5′ end of 20nt guides can impact their activity. The 5′ end of sgCD81i-1 10nt was lengthened with 1-3 additional bases. Either 1 or 2 unpaired bases on the 5′ end resulted in effective repression, while 3 unpaired bases (gcc) completely abrogated repression (FIG. 2A-B). sgCD81i-1 full-length and truncated constructs were tested in A375 (melanoma) cells and demonstrated similar CD81 repression as observed in the Jurkat experiments (FIG. 2C), providing evidence that truncated guides can be active in an additional cellular context. RNA sequencing in A375 showed similar levels of CD81 repression at 20nt and g[9nt] lengths along with additional downregulated targets in the g[9nt] treatment (FIG. 2D). In sum, 5′ truncated guides can direct CRISPRi components to induce repression at target promoters comparable to full-length guides.

### Example 2—Enhancer Disruption with Truncated Guides

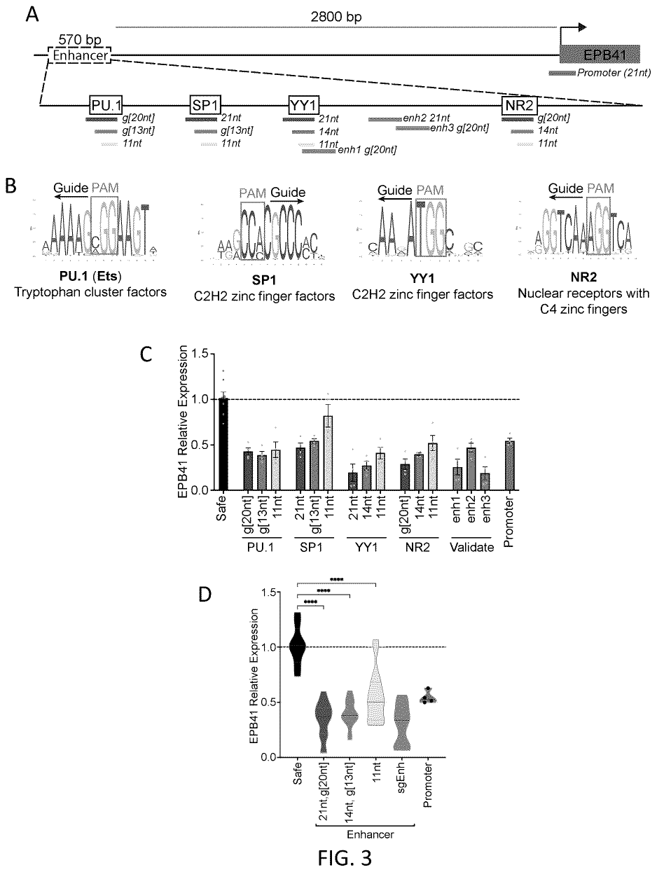

Truncated guides directed toward multiple TF motif sequences in an active enhancer were analyzed. A 570 bp locus with several putative TF binding sites was selected, 2.8 kb upstream of the EPB41 gene (FIG. 3A, chr1:28,883,749-28,884,318). This locus was previously identified as a possible regulator of EPB41 in a K562 CRISPRi screen (Gasperini et al. (2019) Cell, 176(6)). Four TF motifs in this enhancer (PU.1, SP1, YY1 and NR2) were selected, each containing an ideally positioned NGG sequence for the Cas9 protospacer adjacent motif (PAM) (FIG. 3B). TF motifs were positioned at the 3′ end of the guide including the PAM and two truncated versions (g[13nt] or 14nt and 11nt). As shown in Table 3 below, the full-length guides match only the EPB41 enhancer locus while the 11nt guides matched hundreds of additional genomic sites.

TABLE 3
Number of sequence match target sites in the genome (hg38) for the
indicated motif directed guide.

| Target Site | PU.1 | SP1 | YY1 | NR2 |
|---|---|---|---|---|
| g[20 nt], 21nt | 1 | 1 | 1 | 1 |
| g[13 nt], 14nt | 7 | 14 | 11 | 4 |
| 11 nt | 349 | 705 | 463 | 423 |

*Full length guide for PU.1 and NR2 is g[20 nt]; Full length guide for SP1 and YY1 is 21 nt; Truncated guides for PU.1 and SP1 are g[13 nt] and 11 nt; Truncated guides for YY1 and NR2 are 14 nt and 11 nt.

K562 cells were transduced and on-target efficiency measured for EPB41 knockdown by real-time quantitative PCR of EPB41 and compared to 3 guides (enh1-3) identified from the prior screen (Gasperini et al. (2019) Cell, 176(6):1516) as well as a promoter targeting guide (FIG. 3C). EPB41 expression was reduced to levels comparable to the respective 20nt guide in 3 out of 4 11nt guides (PU.1, YY1, NR2) (FIG. 3C). In aggregate, the full-length and truncated guides tested significantly decreased EPB41 expression as compared to safe harbor control (FIG. 3D, one-way ANOVA p<0.0001). The experiment was repeated using a second EPB41 probe with the same results (data not shown). CRISPRi-directed truncated guides can effectively disrupt an enhancer.

### Example 3—a CTCF-Directed Truncated Guide Library

To test the utility of truncated guides for multi-locus TF perturbation, CCCTC-binding factor (CTCF) sites were screened. CTCF is a ubiquitously expressed TF whose role in genomic insulation is dependent on convergently oriented consensus sequences (de Wit et al. (2015) Molecular Cell, 60(4):676-684). Leveraging the 3′ NGG PAM sequence in the CTCF motif (FIG. 4A), a library of 24 10nt guides targeting a total of 13,352 sequence match CTCF binding sites was designed (FIG. 4A-E). Based on CTCF ChIP-seq in Jurkat and A375 cells, approximately half of these sites are CTCF-bound (6,228 in Jurkat cells and 6,140 in A375 cells) and represent 10.8% and 14.0% of all CTCF peaks, respectively (FIG. 4C-E).

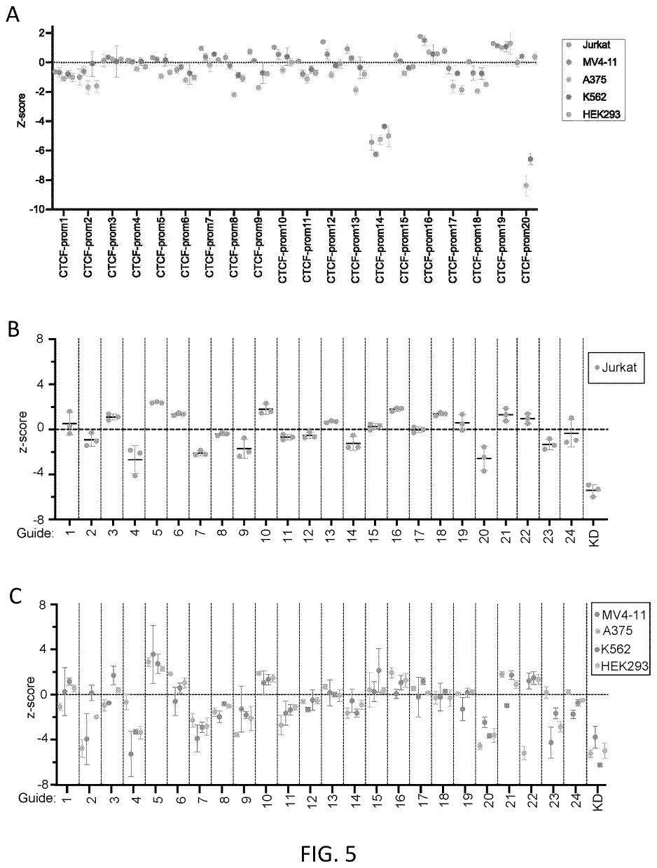

This library was used to test CTCF binding sites, partitioned by guide, ranging from a minimum of 182 sites (sg1) to maximum of 1,123 sites (sg24). As a control the CTCF locus itself was targeted with full-length guides for gene repression (FIG. 5A). Guides were packaged into a lentiviral library, Jurkat cells were transduced near an MOI of 0.5, and cells were collected over 21 days. Over 3 time points, guide enrichment and depletion were measured as a proxy for fitness. The scale of this effect was quantified with a z-score relative to 15 full-length safe harbor guides (see, Methods). Results indicated that most truncated guides were not lethal, as moderate shifts were observed in guide representation relative to Safe Harbor guides (FIG. 5B). A subset of guides resulted in enrichment, suggesting changes that may promote proliferation. Guides sg4 and sg20 were also identified as broadly depleted, though not to the degree of CTCF knockdown. This initial screen provided evidence that certain truncated guides can induce fitness changes in Jurkat cells.

Next the CTCF library was screened in additional cell lines to compare with Jurkat results. In addition to A375 cells, K562 (T-lymphocytes), MV4-11 (AML) and HEK293 were further included as additional models representing diverse cellular contexts for screening. Cells were transduced with the CTCF library and assessed for guide representation after 21 days. Fitness effects in these additional cell models largely recapitulated trends observed in Jurkat cells (FIG. 5C). This could be attributed to invariance of CTCF binding sites across tissues Kim et al. (2007) Cell, 128(6):1231-1245; Vietri Rudan et al. (2015) Cell Reports, 10(8):1297-1309). However, some instances of cell-specific fitness effects was observed, particularly with sg2, sg22, and sg23. While sg2 and sg23 impacted more than one cell line, sg22 was strongly depleted in A375 only.

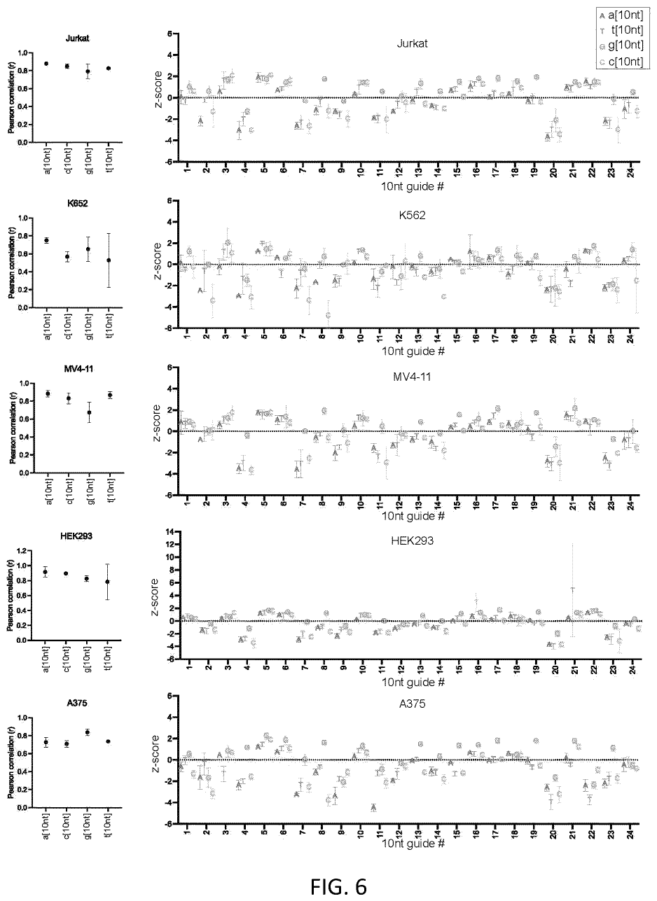

As an additional test of guide-sequence specificity, 11nt guides were screened by adding every base to each 10nt guide in the CTCF library, totaling 96 guides. The additional 5′ base had modest changes on fitness outcomes when compared to the respective 10nt guide outcome (pearson correlations ranged from 0.86 to 0.94 in Jurkat, 0.64 to 0.85 in K562, 0.79 to 0.94 in MV4-11, 0.86 to 0.96 in HEK293, and 0.73 to 0.87 in A375, FIG. 6). This reinforced the prior finding that a single base mismatch was not detrimental to targeting, whereas ≥2 mismatches can disrupt activity (FIG. 2A-B). Testing in 5 cell lines, showed that addition of a single 5′ base to a 10nt guide does not significantly alter guide effects.

### Example 4—Simultaneous Targeting of CTCF Binding Sites

Guide sg4 was selected for further exploration due to its effects on fitness. Guide sg4 targets 357 sites in the genome with 10nt complementarity, termed “perfect match” sites. Perfect match sites were determined regardless of the 5′ guanine present on all guides. Jurkat cells were transduced with sg4 and CRISPRi for 6 or 7 days and CTCF and H3K9me3 ChIP-seq was performed. The analysis revealed a significant drop in CTCF occupancy at perfect match sites (t-test, p-value 5.62×10⁻¹³) (FIGS. 7A-C). CTCF, a strong binder to chromatin, was displaced at many sites simultaneously.

Concurrent with CTCF loss was a significant increase in H3K9me3 signal at perfect match sites (t-test, p-value 1.30×10⁻³⁸) (FIGS. 8A-B). H3K9me3 is a histone mark that indicates KRAB-dCas9 binding and recruitment of repressive proteins (Gilbert et al. (2013) Cell, 154(2):442-451). Two example tracks of perfect match sites depict decreased CTCF binding and concurrent increased H3K9me3 signal (FIGS. 8C-D). These initial findings presented compelling on-target CTCF disruption at multiple sequence match genomic loci with truncated guides.

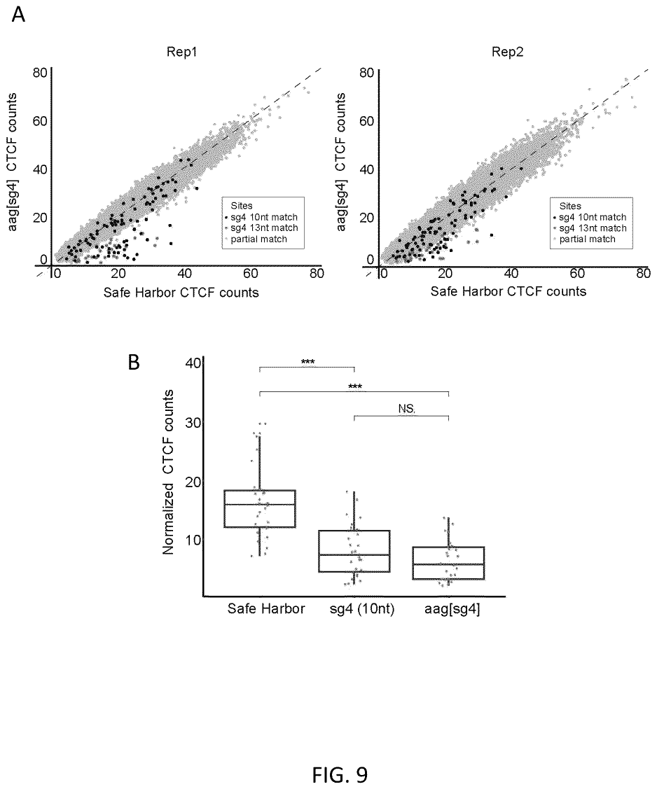

Next multi-locus targeting specificity with a slightly longer spacer sequence was evaluated. A 13nt guide was designed based on the prior findings that 3 mismatched bases disrupt targeting of CD81 TSS (FIG. 2A-B). 3 bases (aag) were appended to the 5′ end of sg4, termed aag[sg4], resulting in a guide with 77 expected perfect match sites. Jurkat cells were transduced with the guide and CRISPRi and processed cells for CTCF ChIP-seq. Examining the 26 CTCF-bound genomic regions targeted by both guides, significantly lower CTCF signal in the aag[sg4] and sg4 samples compared to safe harbor guide were observed (FIGS. 9A-B). Sg4 and aag[sg4] were not significantly different from one another, however a slightly lower mean CTCF signal in aag[sg4] was observed.

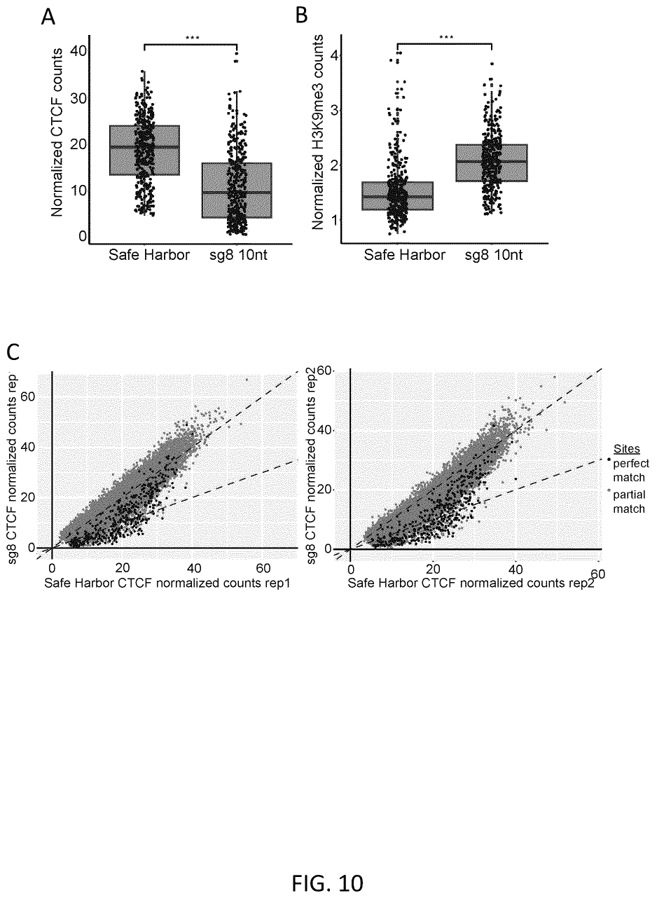

An additional truncated guide from the CTCF library was investigated further for its efficiency. Guide sg8 (10nt) was selected that had 465 sequence match sites and little fitness impact on Jurkat cells. Jurkat cells were transduced with sg8 and CRISPRi and the cells processed for ChIP-seq after 7 days. CTCF binding was severely impacted at sequence match bound sites (t-test, p-value 5.02×10⁻⁴⁷, 411 sites) along with an increase in H3K9me3 signal (t-test, p-value 4.43×10⁻⁴⁶) (FIGS. 10A-C). This complemented the CTCF loss and H3K9me3 gain results observed with sg4. Therefore, truncated guide CRISPRi can deplete CTCF binding events in a bulk population and at a few hundred genomic loci.

Next gene expression effects of CTCF-directed truncated guides was examined. Samples of sg4 and sg8 were collected 7 days post lentiviral transduction and processed for RNA sequencing. Significant genes were determined with dream differential expression analysis (Hoffman & Roussos (2021) Bioinformatics, 37(2):192-201), that compiles confidence from replicate data. Gene expression for sg4 indicated no differential genes (FIGS. 11A and C). However, targeting with sg8 resulted in upregulation of 67 genes (FIGS. 11B and D) which may be explained by one or more sg8 sites resulting in genome reorganization due to lost CTCF-mediated loops (Flavahan et al. (2016) nature, 529(7584):110-114; Hnisz et al. (2016) Science, 351(6280):1454-1458; Liu et al. (2016) Cell, 167(1):233-247; Nora et al. (2017) Cell, 169(5):930-944). Interestingly, the sg8 motif is bound by CTCF at 88.4% of sequence match sites compared to 46.6% for all tested 10nt guides (Table 3). Further investigation is needed to implicate novel enhancer-promoter interactions due to CTCF insulator loss. CTCF disruption with one truncated guide led to no differential gene expression while another guide induced gene upregulation.

### Example 5—Significantly Disrupted CTCF Sites

A comprehensive approach to identify significantly impacted CTCF sites was next pursued. A list of over 880k JASPAR CTCF binding sites in the genome was generated (see, Methods). CTCF ChIP-seq read counts mapping to these regions for sg4 or sg8 were plotted relative to safe harbor controls (FIGS. 7B and 10C). This allowed for proper visualization of both targeted and non-targeted CTCF sites. Dream differential analysis was then applied to determine significant sites. This analysis determined that 64% of Jurkat sg4 sites were CTCF depleted (79 out of 123 perfect match bound sites, p-value<1×10⁻⁵, LFC<0.5) (FIGS. 12A-B). Similarly, dream analysis of sg8 Jurkat samples showed significant loss at 55.7% of CTCF sites (FIG. 12C, 229 of 411 perfect match bound sites, p-value<1×10⁻⁵, LFC<0.5). This data showed that hundreds of CTCF sites can be depleted with truncated guides and over half of targeted CTCF peaks are lost at this scale.

CTCF loss at sites other than perfect match loci was investigated. All putative CTCF binding sites with ≤9nt complementarity to the 10nt guide were collectively termed “partial match” sites. The 880k JASPAR annotated motifs was used for analysis. Partial match sites were analyzed in sg4 containing 1 to 5 mismatches and observed 78 out of 29,908 sites had significant CTCF loss (p-value<1×10⁻⁵, LFC<0.5) (FIGS. 12A-B). Most partial match sites with CTCF loss had a Int mismatch. Motif analysis further illuminated mismatch tolerance positions in the guide at A-T rich bases and the 5′ end for sg4 (FIG. 12D) and sg8 (FIG. 12E). It is noteworthy that most partial match sites remained unaffected by the CRISPRi truncated guides, indicating strong sequence specificity for targeted CTCF sites.

### EMBODIMENTS

Embodiment 1: a high-throughput method for determining functional, non-coding transcriptional regulatory elements, comprising: (a) delivering to a cell a composition comprising: (i) a single guide RNA (sgRNA) or a polynucleotide encoding the sgRNA, wherein the sgRNA comprises a sequence capable of hybridizing with a target sequence, and (ii) an effector protein or one or more nucleotide sequences encoding the effector protein, wherein the effector protein optionally comprises an effector domain; wherein the sgRNA hybridizes to the target sequence and forms a complex with the effector protein; (b) measuring gene expression upon binding of the complex to the target sequence.

Embodiment 2: the method of embodiment 1, wherein the target sequence is a repeated nucleic acid sequence.

Embodiment 3: the method of embodiment 2, wherein the repeated nucleic acid sequence is present in non-coding regulatory element, such as a promoter, an enhancer, a transposable element, a 5′UTR, or an intron.

Embodiment 4: the method of any of embodiment 1-3, wherein the target sequence is a transcription factor binding sequence.

Embodiment 5: the method of any of embodiments 1-4, wherein the transcription factor binding sequence binds a transcription factor from the Ets tryptophan cluster factors family of transcription factors, the C2H2 zinc finger factors family of transcription factors, or the nuclear receptors with C4 zinc fingers family of transcription factors.

Embodiment 6: the method of embodiment 5, wherein the transcription factor is selected from the group consisting of AhR:Arnt, AIRE, alpha-CP1, Alx-4, AML AP-2, AP-2alphaA, AR, AREB6, ATF6, BCL6, Brachyury, CACCC-binding factor, CDP, c-Ets-1 p54, c-Myc, COUP direct repeat 1, CP2/LBP-1c/LSF, c-Rel, CCCTC-binding factor (CTCF), CTF1, deltaEF1, E12, E2F-1, E47, EBF, Egr-1, Egr-3, Elf-1, Elk-1, ER, Ets, ETS2, FXR inverted repeat 1, FXR/RXR-alpha, GABP, GAF, GCM, GLI, GLI1, Hand1:E47, HEB, Helios A, HEN1, HNF4 direct repeat 1, HNF4, COUP, Ikaros, KAISO, LM4_M2, LRF, LRH1, LUN-1, LXR, LXR direct repeat 4, LXR, PXR, CAR, COUP, RAR, Lyf-1, MAF, MIF-1, MIZF, MyoD, myogenin/NF-1, NERF1a, NF-1, NF-AT, NF-kappaB, NF-kappaB (p50), NF-kappaB (p65), NF-muE1, NF-Y, NGFI-C, NRSF, NUR77, Olf-1, P50: RELA-P65, p53, Pax, Pax-1, Pax-6, PEBP, Pitx2, PPAR, PPAR direct repeat 1, PPARalpha:RXRalpha, PPARgamma:RXRalpha, PPARgamma, PU.1, PXR, CAR, LXR, FXR, RBP-Jkappa, RelB:p52 (NF-kappaB), REST, RFX1, Roaz, RORalpha, RORalpha1, RORalpha2, RP58, SAP-1a, SF1, Sp3, SREBP, SREBP-1, SRF, Staf, STAT1, STAT3, STAT5A (homodimer), STAT5B (homodimer), STATx, SZF1-1, TAL1, Tax/CREB, TBX22, TBX5, Tel-2, VDR, YY1, ZBRK1, Zic2, and ZID.

Embodiment 7: the method of any of embodiments 1-6, wherein the sgRNA is 9nt-21nt in length.

Embodiment 8: the method of any of embodiments 1-7, wherein the sgRNA is 9nt-14nt in length.

Embodiment 9: the method of any of embodiments 1-8, wherein the sgRNA is 9nt in length.

Embodiment 10: the method of any of embodiments 1-9, wherein the sgRNA comprises 0-5 mismatched base pairs with the target sequence.

Embodiment 11: the method of any of embodiments 1-10, wherein the effector protein comprises a zinc finger nuclease, a TALEN, a Cas protein, Cpf1 (Cas12), C2c2 (Cas13), Csm/Cmr, C2c6, Csy4, Csm6, C2c2c, dC2c, or Fanzor.

Embodiment 12: the method of any of embodiments 1-11, wherein the method comprises a Clustered Regularly Interspaced Short Palindromic Repeats (CRISPR)-associated (CRISPR-Cas) system.

Embodiment 13: the method of any of embodiments 1-12, wherein the effector domain is selected from the group consisting of: transposase domain, integrase domain, recombinase domain, resolvase domain, invertase domain, protease domain, DNA methyltransferase domain, DNA hydroxylmethylase domain, DNA demethylase domain, histone acetylase domain, histone deacetylases domain, nuclease domain, repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase, and histone tail protease.

Embodiment 14: the method of any of embodiments 1-13, wherein the effector protein comprises a Cas protein fused to a repression domain.

Embodiment 15: the method of embodiment 13 or 14, wherein the repression domain is selected from the group consisting of KRAB, DNMT1, HDAC, DNMT3A, DNMT3A/3L, MeCP2, SID, NuRD, HP1, LSD1, SETDB1, EZH2, or a combination thereof.

Embodiment 16: the method of any of embodiments 1-15, wherein the effector protein comprises a Cas protein fused to an activator domain.

Embodiment 17: the method of any of embodiments 13-16, wherein the activator domain is selected from the group consisting of VP64, p65, VPR (VP64-p65-Rta), p65-HSF1, VPR-RecA, or a combination thereof.

Embodiment 18: the method of any of embodiments 1-17, wherein the effector protein is a catalytically inactive Cas 9 (dCas9).

Embodiment 19: the method of any of embodiments 1-18, wherein the target sequence is a CTCF, PU.1, SP1, YY1, or NR2 binding sequence.

Embodiment 20: the method of any of embodiments 1-19, wherein the sgRNA comprises a nucleotide sequence as set forth in any one of SEQ ID Nos: 1-46 and 50-192.

Embodiment 21: an engineered, non-naturally occurring composition, comprising: a) a single guide RNA (sgRNA) which comprises a sequence capable of hybridizing with a target sequence, or a polynucleotide encoding the sgRNA, and b) an effector protein, or one or more nucleotide sequences encoding the effector protein; wherein the sgRNA hybridizes to said target sequence, and the sgRNA forms a complex with the effector protein; wherein the effector protein optionally comprises an effector domain, wherein the sgRNA is capable of hybridizing multiple transcription factor binding sites.

Embodiment 22: the method of embodiment 21, wherein the transcription factor binding site binds a transcription factor from the Ets tryptophan cluster factors family of transcription factors, the C2H2 zinc finger factors family of transcription factors, or the nuclear receptors with C4 zinc fingers family of transcription factors.

Embodiment 23: the method of embodiment 22, wherein the transcription factor is selected from the group consisting of AhR:Arnt, AIRE, alpha-CP1, Alx-4, AML AP-2, AP-2alphaA, AR, AREB6, ATF6, BCL6, Brachyury, CACCC-binding factor, CDP, c-Ets-1 p54, c-Myc, COUP direct repeat 1, CP2/LBP-1c/LSF, c-Rel, CCCTC-binding factor (CTCF), CTF1, deltaEF1, E12, E2F-1, E47, EBF, Egr-1, Egr-3, Elf-1, Elk-1, ER, Ets, ETS2, FXR inverted repeat 1, FXR/RXR-alpha, GABP, GAF, GCM, GLI, GLI1, Hand1:E47, HEB, Helios A, HEN1, HNF4 direct repeat 1, HNF4, COUP, Ikaros, KAISO, LM4_M2, LRF, LRH1, LUN-1, LXR, LXR direct repeat 4, LXR, PXR, CAR, COUP, RAR, Lyf-1, MAF, MIF-1, MIZF, MyOD, myogenin/NF-1, NERF1a, NF-1, NF-AT, NF-kappaB, NF-kappaB (p50), NF-kappaB (p65), NF-muE1, NF-Y, NGFI-C, NRSF, NUR77, Olf-1, P50: RELA-P65, p53, Pax, Pax-1, Pax-6, PEBP, Pitx2, PPAR, PPAR direct repeat 1, PPARalpha:RXRalpha, PPARgamma:RXRalpha, PPARgamma, PU.1, PXR, CAR, LXR, FXR, RBP-Jkappa, RelB:p52 (NF-kappaB), REST, RFX1, Roaz, RORalpha, RORalpha1, RORalpha2, RP58, SAP-1a, SF1, Sp3, SREBP, SREBP-1, SRF, Staf, STAT1, STAT3, STAT5A (homodimer), STAT5B (homodimer), STATx, SZF1-1, TAL1, Tax/CREB, TBX22, TBX5, Tel-2, VDR, YY1, ZBRK1, Zic2, and ZID.

Embodiment 24: the composition of any of embodiments 21-23, wherein the sgRNA is 9nt-21nt in length.

Embodiment 25: the composition of any of embodiments 21-24, wherein the sgRNA is 9nt-14nt in length.

Embodiment 26: the composition of any of embodiments 21-25, wherein the sgRNA is 9nt in length.

Embodiment 27: the composition of any of embodiments 21-26, wherein the sgRNA comprises 0-5 mismatched base pairs with the target sequence.

Embodiment 28: the composition of any of embodiments 21-27, wherein the effector protein comprises a zinc finger nuclease, a TALEN, a Cas protein Cpf1 (Cas12), C2c2 (Cas13), Csm/Cmr, C2c6, Csy4, Csm6, C2c2c, dC2c, or Fanzor.

Embodiment 29: the composition of any of embodiments 21-28, wherein the composition comprises a Clustered Regularly Interspaced Short Palindromic Repeats (CRISPR)-associated (CRISPR-Cas) system.

Embodiment 30: the composition of any of embodiments 21-29, wherein the effector domain is selected from the group consisting of: transposase domain, integrase domain, recombinase domain, resolvase domain, invertase domain, protease domain, DNA methyltransferase domain, DNA hydroxylmethylase domain, DNA demethylase domain, histone acetylase domain, histone deacetylases domain, nuclease domain, repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase, and histone tail protease.

Embodiment 31: the composition of any of embodiments 21-30, wherein the effector protein comprises a Cas protein fused to a repression domain.

Embodiment 32: the composition of embodiment 30 or 31, wherein the repression domain is selected from the group consisting of KRAB, DNMT1, HDAC, DNMT3A, DNMT3A/3L, MeCP2, SID, NuRD, HP1, LSD1, SETDB1, EZH2, or a combination thereof.

Embodiment 33: the method of any of embodiments 21-32, wherein the effector protein comprises a Cas protein fused to an activator domain.

Embodiment 34: the method of any of embodiments 30-33, wherein the activator domain is selected from the group consisting of VP64, p65, VPR (VP64-p65-Rta), p65-HSF1, VPR-RecA, or a combination thereof.

Embodiment 35: the composition of any of embodiments 21-34, wherein the effector protein is a catalytically inactive Cas 9 (dCas9).

Embodiment 36: the composition of any of embodiments 21-35, wherein the target sequence is a CTCF, PU.1, SP1, YY1, or NR2 binding sequence.

Embodiment 37: the composition of any of embodiments 21-36, wherein the sgRNA comprises a nucleotide sequence as set forth in any one of SEQ ID Nos: 1-46 and 50-192.

Embodiment 38: a single guide RNA (sgRNA) comprising a nucleotide sequence as set forth in any one of SEQ ID NO: 1-46 and 50-192.

Embodiment 39: an adeno-associated virus (AAV) particle comprising the composition of any one of embodiments 21-37, or the sgRNA of embodiment 38.

Embodiment 40: a vector system comprising one or more vectors, wherein the one or more vectors comprises: a) a first expression regulatory element operably linked to a nucleotide sequence encoding an effector protein, or one or more nucleotide sequences encoding the effector protein; and b) a second expression regulatory element operably linked to one or more nucleotide sequences encoding a single guide RNA (sgRNA) comprising a sequence capable of hybridizing to a target sequence, wherein components (a) and (b) are located on same or different vectors, wherein the sgRNA is capable of hybridizing to one or more non-coding transcriptional regulatory elements.

Embodiment 41: the method of embodiment 40, wherein the target sequence is a repeated nucleic acid sequence.

Embodiment 42: the method of embodiment 41, wherein the repeated nucleic acid sequence is present in a promoter, an enhancer, a transposable element, a 5′UTR, or an intron.

Embodiment 43: the vector system of embodiment 42, wherein the target sequence is a transcription factor binding sequence.

Embodiment 44: the method of any of embodiments 40-43, wherein the transcription factor binding sequence binds a transcription factor from the Ets tryptophan cluster factors family of transcription factors, the C2H2 zinc finger factors family of transcription factors, or the nuclear receptors with C4 zinc fingers family of transcription factors.

Embodiment 45: the method of embodiment 44, wherein the transcription factor is selected from the group consisting of AhR:Arnt, AIRE, alpha-CP1, Alx-4, AML AP-2, AP-2alphaA, AR, AREB6, ATF6, BCL6, Brachyury, CACCC-binding factor, CDP, c-Ets-1 p54, c-Myc, COUP direct repeat 1, CP2/LBP-1c/LSF, c-Rel, CCCTC-binding factor (CTCF), CTF1, deltaEF1, E12, E2F-1, E47, EBF, Egr-1, Egr-3, Elf-1, Elk-1, ER, Ets, ETS2, FXR inverted repeat 1, FXR/RXR-alpha, GABP, GAF, GCM, GLI, GLI1, Hand1:E47, HEB, Helios A, HEN1, HNF4 direct repeat 1, HNF4, COUP, Ikaros, KAISO, LM4_M2, LRF, LRH1, LUN-1, LXR, LXR direct repeat 4, LXR, PXR, CAR, COUP, RAR, Lyf-1, MAF, MIF-1, MIZF, MyoD, myogenin/NF-1, NERF1a, NF-1, NF-AT, NF-kappaB, NF-kappaB (p50), NF-kappaB (p65), NF-muE1, NF-Y, NGFI-C, NRSF, NUR77, Olf-1, P50: RELA-P65, p53, Pax, Pax-1, Pax-6, PEBP, Pitx2, PPAR, PPAR direct repeat 1, PPARalpha:RXRalpha, PPARgamma:RXRalpha, PPARgamma, PU.1, PXR, CAR, LXR, FXR, RBP-Jkappa, RelB:p52 (NF-kappaB), REST, RFX1, Roaz, RORalpha, RORalpha1, RORalpha2, RP58, SAP-1a, SF1, Sp3, SREBP, SREBP-1, SRF, Staf, STAT1, STAT3, STAT5A (homodimer), STAT5B (homodimer), STATx, SZF1-1, TAL1, Tax/CREB, TBX22, TBX5, Tel-2, VDR, YY1, ZBRK1, Zic2, and ZID.

Embodiment 46: the vector system of any of embodiments 40-45, wherein the sgRNA is 9nt-21nt in length.

Embodiment 47: the vector system of any of embodiments 40-46, wherein the sgRNA is 9nt-14nt in length.

Embodiment 48: the vector system of any of embodiments 40-47, wherein the sgRNA is 9nt in length.

Embodiment 49: the vector system of any of embodiments 40-48, wherein the sgRNA comprises 0-5 mismatched base pairs with the target sequence.

Embodiment 50: the vector system of any of embodiments 40-49, wherein the effector protein comprises a zinc finger nuclease, a TALEN, a Cas protein, Cpf1 (Cas12), C2c2 (Cas13), Csm/Cmr, C2c6, Csy4, Csm6, C2c2c, dC2c, or Fanzor.

Embodiment 51: the vector system of any of embodiments 40-50, wherein the method comprises a Clustered Regularly Interspaced Short Palindromic Repeats (CRISPR)-associated (CRISPR-Cas) system.

Embodiment 52: the vector system of any of embodiments 40-51, wherein the effector domain is selected from the group consisting of: transposase domain, integrase domain, recombinase domain, resolvase domain, invertase domain, protease domain, DNA methyltransferase domain, DNA hydroxylmethylase domain, DNA demethylase domain, histone acetylase domain, histone deacetylases domain, nuclease domain, repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase, and histone tail protease.

Embodiment 53: the vector system of any of claims 40-52, wherein the effector protein comprises a Cas protein fused to a repression domain.

Embodiment 54: the vector system of embodiment 52 or 53, wherein the repression domain is selected from the group consisting of KRAB, DNMT1, HDAC, DNMT3A, DNMT3A/3L, MeCP2, SID, NuRD, HP1, LSD1, SETDB1, EZH2, or a combination thereof.

Embodiment 55: the method of any of embodiments 40-54, wherein the effector protein comprises a Cas protein fused to an activator domain.

Embodiment 56: the method of any of embodiments 52-55, wherein the activator domain is selected from the group consisting of VP64, p65, VPR (VP64-p65-Rta), p65-HSF1, VPR-RecA, or a combination thereof.

Embodiment 57: the vector system of any of embodiments 40-56, wherein the effector protein is a catalytically inactive Cas 9 (dCas9).

Embodiment 58: the vector system of any of embodiments 40-57, wherein the target sequence is a CTCF, PU.1, SP1, YY1, or NR2 binding sequence.

Embodiment 59: the vector system of any of embodiments 40-58, wherein the sgRNA comprises a nucleotide sequence as set forth in any one of SEQ ID Nos: 1-46 and 50-192.

Embodiment 60: an isolated cell comprising the composition of any one of embodiments 21-37, the sgRNA of embodiment 38, the AAV particle of embodiment 39, or the vector system of embodiments 40-59.

Embodiment 61: an in vitro or ex vivo host cell or cell line or progeny thereof comprising the composition of any one of embodiments 21-37, the sgRNA of embodiment 38, the AAV particle of embodiment 39, or the vector system of embodiments 40-59.

Embodiment 62: a method for screening for functional non-coding transcriptional regulatory elements in a cell, the method comprising delivering to the cell the composition of any one of embodiments 21-37, the sgRNA of embodiment 38, the AAV particle of embodiment 39, or the vector system of embodiments 40-59, wherein the effector protein forms a complex with the sgRNA and upon binding of the complex to a target sequence, a modification in gene expression occurs.

Embodiment 63: the method of embodiment 62, wherein gene expression is increased or decreased upon binding of the complex to a target sequence.

What is claimed is:

1. A high-throughput method for determining functional, non-coding transcriptional regulatory elements, comprising:
(a) delivering to a cell a composition comprising:
(i) a single guide RNA (sgRNA) or a polynucleotide encoding the sgRNA, wherein the sgRNA comprises a sequence capable of hybridizing with a target sequence, and
(ii) an effector protein or one or more nucleotide sequences encoding the effector protein;
wherein the sgRNA hybridizes to the target sequence and forms a complex with the effector protein; and
(b) measuring gene expression upon binding of the complex to the target sequence.

2. The method of claim 1, wherein the target sequence is present in a non-coding regulatory element, the target sequence is a repeated nucleic acid sequence, or the target sequence is a transcription factor binding sequence.

3. The method of claim 2, wherein the transcription factor binding sequence binds a transcription factor from the Ets tryptophan cluster factors family of transcription factors, the C2H2 zinc finger factors family of transcription factors, or the nuclear receptors with C4 zinc fingers family of transcription factors.

4. The method of claim 1, wherein the sgRNA is 9 nt-21 nt in length, the sgRNA is less than 14 nt in length, or the sgRNA comprises 0-5 mismatched base pairs with the target sequence.

5. The method of claim 1, wherein the sgRNA comprises a nucleotide sequence as set forth in any one of SEQ ID NOs: 1-46 and 50-192.

6. The method of claim 1, wherein the effector protein comprises a zinc finger nuclease, a TALEN, a Cas protein, Cpf1 (Cas12), C2c2 (Cas13), Csm/Cmr, C2c6, Csy4, Csm6, C2c2c, dC2c, a Fanzor, a Cas protein fused to a repressor domain, a Cas protein fused to an activator domain, or a catalytically inactive Cas 9 (dCas9).

7. The method of claim 1, wherein the method further comprises a Clustered Regularly Interspaced Short Palindromic Repeats (CRISPR)-associated (CRISPR-Cas) system.

8. The method of claim 1, wherein the effector protein comprises an effector domain selected from the group consisting of: a transposase domain, an integrase domain, a recombinase domain, a resolvase domain, an invertase domain, a protease domain, a DNA methyltransferase domain, a DNA hydroxylmethylase domain, a DNA demethylase domain, a histone acetylase domain, a histone deacetylases domain, a nuclease domain, a repressor domain, an activator domain, a nuclear-localization signal domain, a transcription-regulatory protein domain, a transcription complex recruiting domain, a cellular uptake activity associated domain, a nucleic acid binding domain, an antibody presentation domain, a histone modifying enzyme, a recruiter of histone modifying enzymes; an inhibitor of histone modifying enzymes, a histone methyltransferase, a histone demethylase, a histone kinase, a histone phosphatase, a histone ribosylase, a histone deribosylase, a histone ubiquitinase, a histone deubiquitinase, a histone biotinase, and a histone tail protease.

9. The method of claim 6, wherein the repressor domain is selected from the group consisting of: KRAB, DNMT1, HDAC, DNMT3A, DNMT3A/3L, MeCP2, SID, NuRD, HP1, LSD1, SETDB1, EZH2, or a combination thereof; or the activator domain is selected from the group consisting of: VP64, p65, VPR (VP64-p65-Rta), p65-HSF1, VPR-RecA, or a combination thereof.

10. The method of claim 2, wherein the non-coding regulatory element is a promoter, an enhancer, a transposable element, a 5′UTR, or an intron.

11. The method of claim 1, wherein the target sequence is a CTCF, PU.1, SP1, YY1, or NR2 binding sequence.

12. An engineered, non-naturally occurring composition comprising:
a) a single guide RNA (sgRNA) or a polynucleotide encoding the sgRNA, said sgRNA comprising a sequence capable of hybridizing with a target sequence, and
b) an effector protein or one or more nucleotide sequences encoding the effector protein;
wherein the sgRNA hybridizes to said target sequence, and the sgRNA forms a complex with the effector protein; and
wherein the sgRNA is capable of hybridizing multiple transcription factor binding sites.

13. The composition of claim 12, wherein at least one of the multiple transcription factor binding sites binds a transcription factor from the Ets tryptophan cluster factors family of transcription factors, the C2H2 zinc finger factors family of transcription factors, or the nuclear receptors with C4 zinc fingers family of transcription factors.

14. The composition of claim 12, wherein the sgRNA is 9 nt-21 nt in length, the sgRNA is less than 14 nt in length, the sgRNA comprises 0-5 mismatched base pairs with the target sequence, or the sgRNA comprises a nucleotide sequence as set forth in any one of SEQ ID Nos: 1-46 and 50-192.

15. The composition of claim 12, wherein the effector protein comprises a zinc finger nuclease, a TALEN, a Cas protein Cpf1 (Cas12), C2c2 (Cas13), Csm/Cmr, C2c6, Csy4, Csm6, C2c2c, dC2c, a Fanzor, a Cas protein fused to a repression domain, or Cas protein fused to an activator domain.

16. The composition of claim 12, further comprising a Clustered Regularly Interspaced Short Palindromic Repeats (CRISPR)-associated (CRISPR-Cas) system.

17. The composition of claim 12, wherein the effector protein comprises an effector domain selected from the group consisting of: a transposase domain, an integrase domain, a recombinase domain, a resolvase domain, an invertase domain, a protease domain, a DNA methyltransferase domain, a DNA hydroxylmethylase domain, a DNA demethylase domain, a histone acetylase domain, a histone deacetylases domain, a nuclease domain, a repressor domain, an activator domain, a nuclear-localization signal domain, a transcription-regulatory protein domain, a transcription complex recruiting domain, a cellular uptake activity associated domain, a nucleic acid binding domain, an antibody presentation domain, a histone modifying enzymes, a recruiter of histone modifying enzymes; an inhibitor of histone modifying enzymes, a histone methyltransferase, a histone demethylase, a histone kinase, a histone phosphatase, a histone ribosylase, a histone deribosylase, a histone ubiquitinase, a histone deubiquitinase, a histone biotinase, and a histone tail protease.

18. A single guide RNA (sgRNA) comprising a nucleotide sequence as set forth in any one of SEQ ID NO: 1-46 and 50-192.

19. An adeno-associated virus (AAV) particle comprising the sgRNA of claim 18.

20. A method for screening for functional non-coding transcriptional regulatory elements in a cell, the method comprising:
delivering the composition of claim 12 to the cell,
forming a complex with the effector protein and the sgRNA, and
binding the complex to a target sequence to cause a modification in gene expression.
